(19) United States
(12) Patent Application Publication
(10) Pub. No.: US 2024/0142419 A1
(43) Pub. Date: May 2, 2024

(54) PROOF OF WORK FOR CARBON REDUCTION/RECLAMATION

(71) Applicant: RECARBON, INC., Santa Clara, CA (US)

(72) Inventors: John Kirt Liu, Hillsborough, CA (US); Jae Mo Koo, Palo Alto, CA (US)

(73) Assignee: RECARBON, INC., Santa Clara, CA (US)

(21) Appl. No.: 18/494,728

(22) Filed: Oct. 25, 2023

Related U.S. Application Data
(60) Provisional application No. 63/381,283, filed on Oct. 27, 2022.

(51) Int. Cl. G01N 33/00; G01D 4/00; G01D 21/02

(52) U.S. Cl. CPC G01N 33/0014; G01D 4/008; G01D 21/02; G01N 33/004

(57) ABSTRACT

A token adjustment system may include a carbon conversion device operable to generate a syngas from a greenhouse gas and a processing device. The processing device may be operable to receive one or more measurements. The one or more measurements may include one or more of an input flow measurement received from an input flow sensor, an output flow measurement received from an output flow sensor, or a utility measurement received from a utility sensor. The processing device may be operable to compute a carbon balance, a carbon intensity, or a carbon adjustment based on the one or more measurements. The processing device may be operable to cause a token to be generated or consumed based on the carbon balance, the carbon intensity, or the carbon adjustment.

### CROSS REFERENCE TO RELATED APPLICATIONS

This U.S. patent application claims priority to Provisional Patent Application 63/381,283 filed on Oct. 27, 2022. The disclosure of this prior application is considered part of the disclosure of this application and is hereby incorporated by reference in its entirety.

### TECHNICAL FIELD

The present disclosure generally relates to proof of work for carbon reduction/reclamation, including such proof of work in conjunction with a plasma reaction system.

### BACKGROUND

Carbon and carbon dioxide are elements found in all living things. However, excess carbon dioxide and methane, as a by-product of human activity, contributes to climate change. As technology is developed to use carbon emissions as a resource, those technologies may provide a cleaner, greener, and more decarbonized economy. To help encourage carbon reduction and reclamation activities, some governments have offered carbon sequestration tax credits to provide economic incentives. Other innovations to drive carbon reduction include voluntary carbon markets that permit entities to buy and sell carbon credits. Typically, carbon credits are granted by governments as an “allowance” for carbon emissions. Unused credits may be sold to other entities.

The subject matter claimed in the present disclosure is not limited to embodiments that solve any disadvantages or that operate only in environments such as those described above. Rather, this background is only provided to illustrate one example technology area where some embodiments described in the present disclosure may be practiced.

### SUMMARY

A token adjustment system may include a carbon conversion device operable to generate a syngas from a greenhouse gas and a processing device. The processing device may be operable to receive one or more measurements. The one or more measurements may include one or more of an input flow measurement received from an input flow sensor, an output flow measurement received from an output flow sensor, an energy measurement from an energy sensor, or a utility measurement received from a utility sensor. The processing device may be operable to compute one or more of a carbon balance, a carbon intensity, or a carbon adjustment based on the one or more measurements. The processing device may be operable to cause a token to be generated or consumed based on the one or more of the carbon balance, the carbon intensity, or the carbon adjustment.

A method for adjusting tokens may include detecting one or more measurements including one or more of an input flow measurement received from an input flow sensor, an output flow measurement received from an output flow sensor, an energy measurement from an energy sensor, or a utility measurement received from a utility sensor. The method may include computing one or more of a carbon balance, a carbon intensity, or a carbon adjustment based on the one or more measurements. The method may include causing a token to be adjusted based on the one or more of the carbon balance, the carbon intensity, or the carbon adjustment.

A device may be operable to adjust tokens. The device may include a sensor operable to measure one or more metrics comprising one or more of an environmental input metric, an environmental output metric, or an environmental energy metric. The device may include a processing device operable to compute an environmental effect based on the one or more metrics and cause a token to be adjusted based on the environmental effect.

### BRIEF DESCRIPTION OF THE DRAWINGS

Example embodiments will be described and explained with additional specificity and detail through the accompanying drawings in which:

FIG. 1 illustrates an example of a system for processing inputs into outputs with an environmental effect.

FIG. 2 illustrates an example process flow for token adjustment.

FIG. 3 illustrates an example of a decarbonization system for converting input greenhouse gases to decarbonized syngas.

FIG. 4 illustrates an example decarbonization system for converting input greenhouse gases to decarbonized products.

FIG. 5 illustrates an example of a plasma reaction system that includes a plasma chamber and an ancillary reaction chamber.

FIG. 6 illustrates an example of a plasma reaction system that includes an ancillary reaction chamber and an integrated reformer connected in series.

FIG. 7 illustrates an example process flow for token adjustment.

FIG. 8 illustrates an example process flow for token adjustment.

FIG. 9 illustrates an example process flow for token adjustment.

FIG. 10 illustrates a token adjustment process system for converting energy and utility input and a feed to a product.

FIG. 11 is an example process flow for token adjustment.

FIG. 12 is an example process flow for token adjustment.

FIG. 13 is an example process flow for token adjustment.

FIG. 14 is an example system for generating a synthesis gas stream using microwave power, a first gas stream, a second gas stream, and a third gas stream.

FIG. 15 is an example of a plasma carbon conversion unit.

FIG. 16A illustrates a diagram of a plasma reaction system including a plasma reaction chamber and an ancillary reaction chamber connected in parallel to a group of integrated reformers according to an example.

FIG. 16B illustrates a diagram of a plasma reaction system including a group of plasma reaction chambers connected in series to a group of ancillary reaction chambers, which are connected to an integrated reformer according to an example.

FIG. 16C illustrates a diagram of a plasma reaction system including a group of plasma reaction chambers connected in series to a group of ancillary reaction chambers, which are connected to an integrated reformer according to an example.

FIG. 16D illustrates a diagram of a plasma reaction system including a group of plasma reaction chambers connected to an ancillary reaction chamber which is connected to an integrated reformer according to an example.

FIG. 16E illustrates a diagram of a plasma reaction including a group of plasma reaction chambers connected to an ancillary reaction chamber which is connected to an integrated reformer, which is connected to a group of ancillary reaction chambers, according to an example.

FIG. 16F illustrates a diagram of a plasma reaction system including a group of plasma reaction chambers connected to an ancillary reaction chamber which is connected to an integrated reformer, which is connected to a group of ancillary reaction chambers at different inlets, according to an example.

FIG. 17 illustrates a diagram of a system for carbon dioxide utilization using a plasma reaction system according to an example.

FIG. 18 illustrates a diagram of a system for synthesizing hydrogen gas and carbon monoxide using a plasma reaction system according to an example.

FIG. 19 illustrates a diagram of a system for converting biogas into hydrogen gas using a plasma reaction system according to an example.

FIG. 20 illustrates an integrated reformer according to an example.

FIG. 21 illustrates an example computing system that may be used for token adjustment, all arranged in accordance with some examples of the present disclosure.

### DETAILED DESCRIPTION

Innovations to drive carbon reduction include voluntary carbon markets that permit entities to buy and sell carbon credits. Typically, carbon credits are granted by governments as an “allowance” for carbon emissions. Unused credits may be sold to other entities. Under some systems, a carbon credit is a permit that represents 1 ton of carbon dioxide removed from the atmosphere. Any standard of measurement or value assignment may be used for the carbon credit.

Determining whether a carbon reduction process has resulted in any carbon reduction may depend on a few different factors. First, an adequate number of the operations of the process may be accounted for. Second, validating whether the sensing and/or measurement associated with the operations of the process have been included. Third, accounting for all of the relevant inputs (e.g., the carbon input and also the energy and utility input) and weighing those inputs against the outputs (e.g., the carbon reduction output and the net reduction in carbon). Even when these factors are taken into account, a system for weighing these factors and providing proof of work based on these factors would be useful.

Systems, methods, and devices may be used for token adjustment according to carbon balance, carbon intensity, or carbon adjustment. In one example, a token adjustment system may include a carbon conversion device operable to generate decarbonized syngas or other products from a greenhouse gas and a processing device. The processing device may be operable to receive one or more measurements. The one or more measurements may include one or more of an input flow measurement received from an input flow sensor, an output flow measurement received from an output flow sensor, or a utility measurement received from a utility sensor. The processing device may be operable to compute a carbon balance based on the one or more measurements. The processing device may be operable to cause a token to be generated or consumed based on the carbon balance.

An example method operable to adjust tokens may include detecting one or more measurements including one or more of an input flow measurement received from an input flow sensor, an output flow measurement received from an output flow sensor, or a utility measurement received from a utility sensor. The method may include computing a carbon balance based on the one or more measurements. The method may include causing a token to be adjusted based on the carbon balance.

A device operable to adjust tokens may include: a sensor operable to measure one or more metrics including one or more of an environmental input metric, an environmental output metric, or an environmental energy metric, and a processing device. The processing device may be operable to compute an environmental effect based on the one or more metrics, and cause a token to be adjusted based on the environmental effect.

The technology disclosed herein may be used to adjust tokens (e.g., generate tokens or consume tokens) that may be used as carbon credits (e.g., to create tokenized carbon credits or tokenized carbon debits), for governments granting carbon credits or deducting carbon credits, or for any other purpose. The tokens may be backed by a “proof of work” that represents that an actual and verifiable environmental effect (e.g., a benefit or detriment) has happened that backs the token. In general, proof of work may be a form of cryptographic proof in which one party may prove to other parties that a certain amount of a specific computational effort has been expended, and in this case, a proof that work has been performed to impact the environment (e.g., an environmental benefit or environmental detriment).

Verifiers may subsequently confirm this expenditure with minimal effort on their part. For systems relating to carbon (e.g., carbon recapture, reclamation, reduction, or the like), the proof of work may sometimes be referred to as “proof of carbon work.” The tokens adjusted (e.g., generated or consumed) may be sold and purchased, such as on voluntary carbon markets. In some instances, the tokens may be used as, or converted to, carbon credits. Additionally or alternatively, the proof of work may be specific to a particular source and may represent work or operations done by a particular system or machine, and/or at a particular location.

Proof of work may be used when the work is beneficial to the environment, rather than a net consumer of energy. Many blockchain participants use significant energy consumption for “mining” and proof of work which often carries a significant environmental cost due to the energy used by dedicated and expensive hardware as blockchain participants carry out complex calculations used for proof of work. Using techniques of the present disclosure, proof of work may be combined with environmentally beneficial actions and a blockchain may be used to keep a record of each environmentally beneficial activity performed.

Reference will now be made to the drawings to describe various aspects of example embodiments of the invention. It is to be understood that the drawings are diagrammatic and schematic representations of such example embodiments, and are not limiting of the present invention, nor are they necessarily drawn to scale.

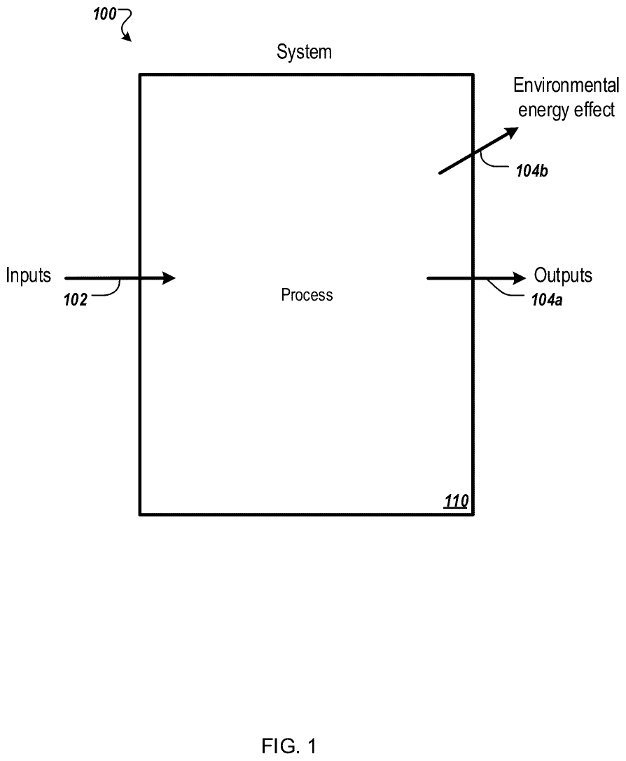

As illustrated in FIG. 1, an example system 100 is provided herein that provides a process 110 to perform environmentally beneficial work that may be used to generate tokens. Any system or technique or calculation may be employed to generate the tokens, so long that the system or technique or calculation employed is verifiable. An environmental effect (e.g., environmentally beneficial action or result or environmentally detrimental action or result) may be used to adjust a token (e.g., generate a token in the case of an environmentally beneficial action or result or consume a token in the case of an environmentally detrimental action or result). For example, in a system that uses multiple reactors in series, a downstream reactor may use some heat from an upstream reactor, which may mean fewer fossil fuels are burned to generate heat for the downstream reactor. An amount of heat saved by the downstream reactor by using the heat from the upstream reactor may be tokenized.

In any example system 100, inputs 102 (e.g., which may be measured, using a sensor, as an environmental input metric) may be known, measured, and/or calculated. Similarly, outputs 104a (e.g., which may be measured as an environmental output metric using a sensor) may be known, measured, and/or calculated. Similarly, an environmental energy effect 104b (e.g., which may be measured, using a sensor, as an output environmental energy effect metric) may be known, measured, and/or calculated. The environmental input metric may a metric that measures an environmental effect of inputs 102. The environmental output metric may be a metric that measures an environmental effect of outputs 104a. The environmental energy effect 104b may be a metric that measures an environmental effect of energy and utility used to generate the outputs 104a from the inputs 102.

In addition to the work itself, the inputs 102, the outputs 104a, and the environmental energy effect 104b may be used for the proof of work. For example, in a plasma reaction system, inputs 102 and input parameters as well as energy and utility may be compared to outputs 104a and output parameters (e.g., materials, chemicals, temperatures, etc.) to demonstrate an environmental effect (e.g., a net environmental benefit, such as through carbon reduction and/or reclamation). Through such a system 100, the amount of carbon reduction and/or reclamation is known. A token may be generated, such as to represent a unit amount of the environmental effect (e.g., net environmental benefit). For example, a particular amount of carbon reduction may represent a token value. The inputs 102 and the outputs 104a, when verifiable, may be used as the proof of work for the token. In some examples, the token may be validated using location data.

The token may be generated or consumed based on the environmental effect. When the environmental output metric is greater than the combined total of the environmental input metric and the environmental energy metric, then the token may be generated (because a net environmental benefit has occurred). When the environmental input metric and the environmental energy metric is greater than the environmental output metric, then the token may be consumed (because a net environmental detriment has occurred).

While the present disclosure is described in the context of a “proof of work” system and related blockchain, any type of “proof” system and blockchain may be used, including a “proof of space,” “proof of stake,” “proof of space and time,” “proof of decarbonization,” “proof of carbon reduction,” “proof of carbon reformation,” or the like.

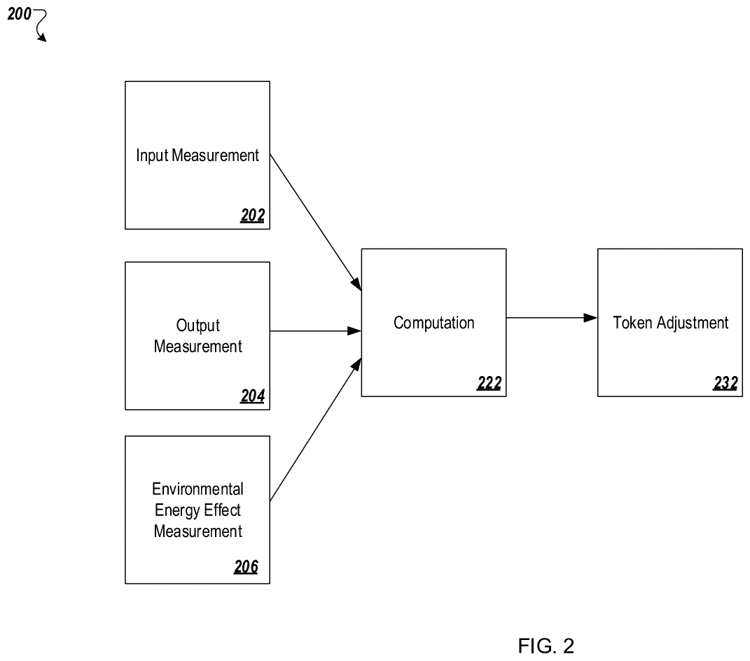

As illustrated in FIG. 2, benefits may be tokenized using the process 200. Input measurement 202, output measurement 204, and environmental energy effect measurement 206 may be used in a computation 222 (e.g., of a carbon balance based on an environmental effect) to facilitate token adjustment 232 (i.e., token generation or token consumption). For example, the process 200 may create a cryptocurrency to tokenize the benefits provided through use of the process 200. The cryptocurrency may be bought or sold, and may be used for carbon offsets. When confidence intervals are used to verify, validate, or prove work, the reward may be increased as the confidence interval increases. A result of the token adjustment 232 may be stored in a record or ledger, such as a blockchain. Any token generation may be recorded in a blockchain and any token consumption may be recorded in a blockchain.

When a blockchain is used to record the token generation and/or the token consumption, the validity of the token generation and/or the token consumption may use one or more of a proof-of-work scheme (e.g., using one or more hashing algorithms such as SHA-256, scrypt, CryptoNote, Blake, SHA-3, X11, or the like) or a proof-of-stake scheme (e.g., Byzantine fault tolerance-based, chain-based, committee-based, delegated, and/or liquid), or any other suitable proof system.

The result of the token adjustment 232 may be stored in any other suitable record or ledger. A cryptocurrency wallet may be used to store the result of the token adjustment 232. For example, token consumption may be recorded by using a private key stored in a cryptocurrency wallet and/or token generation (e.g., token receiving) may be recorded by using a public key stored in the cryptocurrency wallet. Alternatively or in addition, centralized databases (e.g., cryptocurrency market databases) may be used to store the result of the token adjustment 232.

The result of the token adjustment 232 may be stored on-chain (e.g. on a blockchain) or off-chain (e.g., using a centralized hosting provider and/or using a decentralized hosting provider such as the InterPlanetary File System (IPFS)). When stored off-chain, the token (e.g., a non-fungible token (NFT)) may include a smart contract that may point to the location where the token (e.g., the NFT) is stored.

The process may facilitate token adjustment 232 (e.g., token generation) based in part on contracted offsets with third parties. In some reformation or recapture methods, some methods or techniques may provide a more favorable or appealing carbon footprint when compared to other methods or techniques, and the favorability may be at least in part due to a granted offset. The magnitude of the granted offset, in some instances, may be set by an independent organization or government in order to encourage certain types of activities, such as certain methods or techniques with a high benefit to the environment. Tokens may be adjusted (e.g., generated) based on such a granted offset and the amount of related tokens may vary when the magnitude offset also varies.

There may be a “credit” (e.g., a token adjustment 232) provided for environmentally-friendly inputs. For example, when a system is used to manufacture jet fuel, and the input is a bio source, then there may be a credit for that type of input (e.g., the token adjustment 232 may be a token generation). When a fossil fuel is used, there may not be a credit (e.g., the token adjustment 232 may include a token consumption). Alternatively or in addition, there may not be a credit for environmentally-friendly inputs and there may be a penalty for environmentally detrimental inputs. Alternatively or in addition, there may be a penalty corresponding to supplying the input to the system because carbon may be emitted when supplying the input to the system. Alternatively or in addition, there may be a combination of credits (e.g., the token adjustment 232 may include a token generation) and penalties (e.g., the token adjustment 232 may include a token consumption). For example, some methods that emit carbon dioxide using steam methane reforming (SMR) (e.g., using natural gas and water to produce syngas and CO₂ emission) may result in a penalty.

In an example, proof of work to facilitate token adjustment may include a location-based component to additionally validate that the work was performed. For example, a geographical location measurement may be taken (e.g., a global positioning system (GPS) location) which may coincide with a location of a particular system (e.g., a carbon recapture plant). The proof of work may be in real-time and may represent work performed in real-time. Tokens may be adjusted (e.g., generated or consumed) in real time as the work is being performed.

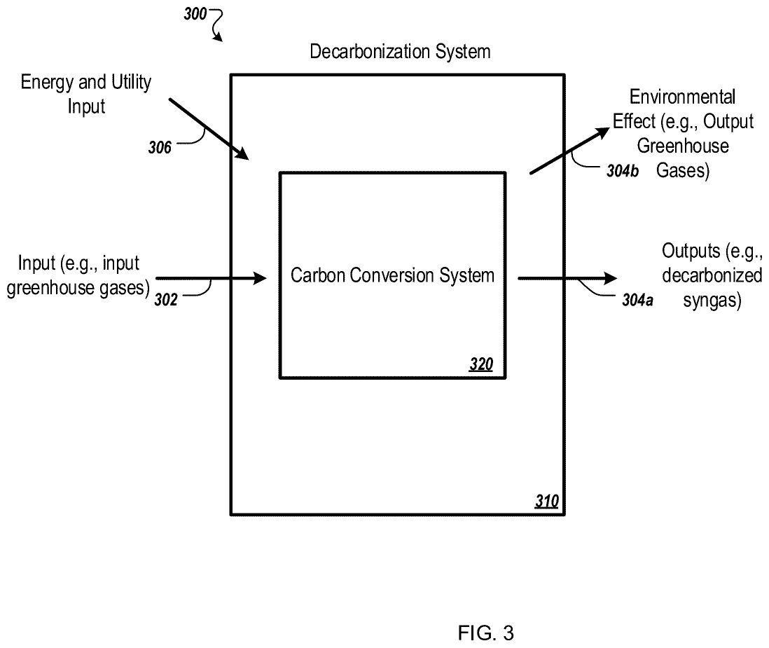

As illustrated in FIG. 3, the disclosed technology may be used with any system 300 that provides environmental benefits, including in a decarbonization system 310 which may include a carbon conversion system 320 (e.g., a plasma reaction system that may use atmospheric microwave plasma) to efficiently convert input 302 (e.g., input greenhouse gases such as CO₂ and CH₄, or natural gas) into outputs 304b (e.g., decarbonized syngas, such as H₂ and CO) and with an environmental effect (e.g., output greenhouse gases 304b). The system 300 may be deployed anywhere, including into existing or new CO₂ emitting sites, which may include biogas, wastewater treatment, steel, cement, petrochemical, power plants, or the like.

Utilizing CO₂ as a carbon source has the public benefit of reducing CO₂ emissions as well as saving natural resources. The energy and utility input 306 (e.g., electricity, heat, water, cooling, heating, drying, and drain) may be supplied from renewable sources, such as solar and wind. With electricity from renewable sources, the carbon intensity of the product may be minimized.

In instances where multiple types of environmentally beneficial systems are used, tokens may be adjusted (e.g., generated or consumed) on a system/aggregate level and/or on a component basis (or based on groups of components). Further, “green” or “renewable” additions to the system, such as solar and/or wind energy sources may be used in the token adjustment (e.g., generation or consumption) process.

The system 300 (e.g., plasma reaction system) may include any number of sensors and any type of sensor to measure inputs (e.g., input measurement 202 as illustrated in FIG. 2) and outputs (e.g., output measurement 204 as illustrated in FIG. 2) and facilitate computation (e.g., as illustrated in 222 in FIG. 2). The resulting sensor measurements and/or computations may be used for proof of work and/or token adjustment (e.g., token adjustment 232 as illustrated in FIG. 2, which may be token generation or consumption). For example, the system 300 (e.g., plasma reaction system) may include any number of chambers, including plasma chambers, non-plasma chambers, ancillary reaction chamber, integrated reformers, or the like. Various sensors may be positioned throughout the system 300 to collect data of the system 300.

The input 302 (which may be input greenhouse gases 302) may include any suitable input including a feed that may include one or more of CO₂, CH₄, O₂, hydrocarbons, or the like. In one example, the feed may include biogas (e.g., CO₂+CH₄) which may be received from any suitable source (e.g., landfill gas, waste treatment, biomass gasification, or the like). Alternatively or in addition, the feed may include CO₂ which may be received from any suitable source (e.g., by capture, from a tank, or the like). Alternatively or in addition, the feed may include natural gas (e.g., CH₄) which may be received from any suitable source (e.g., pipeline, tank, or the like). Alternatively or in addition, the feed may include O₂ which may be received from any suitable source (e.g., from an O₂ generator, a tank, or the like). Alternatively or in addition, the feed may include hydrocarbon gases which may be received e.g., from coal gasification. The feed may include any suitable combination of inputs.

The output 304a may include products which may include one or more of H₂, syngas (Hz and CO), CO₂, steam, heat, water, or the like. An environmental effect 304b may occur (e.g., the amount of CO₂ released as an output 304a may be increased or reduced when compared to the amount of CO₂ provided as an input 302). The output syngas ratio (H₂:CO) may be from about 0.5:1 to about 3:1.

Various parameters may be used including input parameters, output parameters, and operating parameters. The input parameters may include, without limitation, one or more of: flowrate, composition, pressure, temperature, and/or materials. The output parameters may include, without limitation, one or more of: flowrate, composition, pressure, temperature, and/or materials. The energy and utility parameters may include, without limitation, one or more of: electricity consumption, fuel consumption, heat consumption, coolant flowrate, pressure, temperature, coolant materials, drain treatment flowrate, steam consumption, steam generation, particulate removal rate, or the like. The operating parameters may include, without limitation, one or more of temperature, pressure, volume, sound, humidity, elevation, ventilation, power, fuel, or the like.

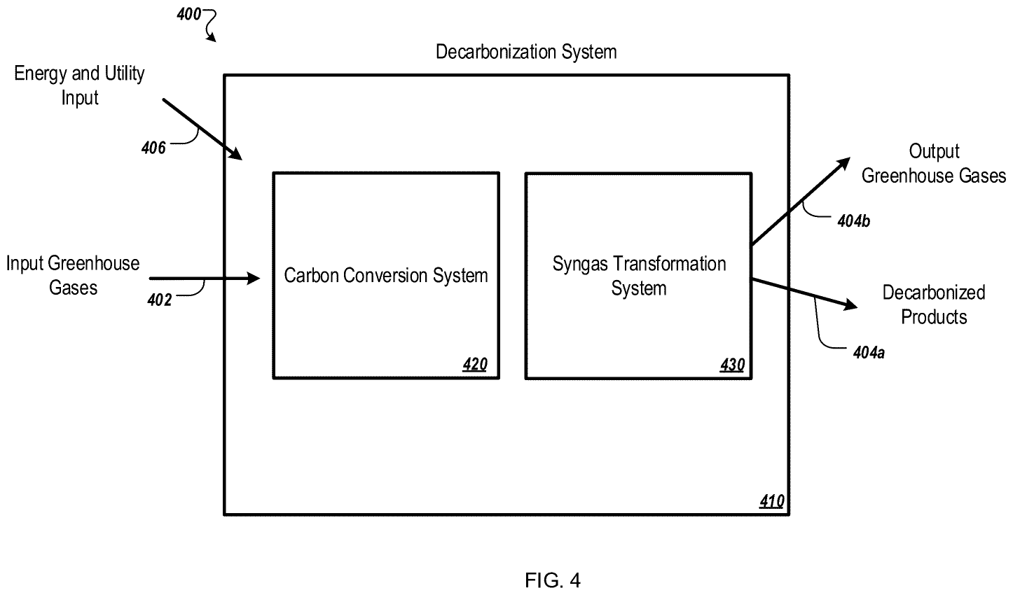

As illustrated in FIG. 4, a system 400 (e.g., a decarbonization system 410) may include a carbon conversion system 420 (which may be a plasma reaction system) and a syngas transformation system 430 (e.g., a syngas transformation device). The decarbonization system 410 may be operable to receive an energy and utility input 406 and input greenhouse gases 402 (e.g., CO₂+CH₄). The decarbonization system 410 may be operable to output decarbonized products 404a and output greenhouse gases 404b. The input greenhouse gases 402, the decarbonized products 404a, the output greenhouse gases 404b, and the energy and utility input 406 may be measured to compute an environmental effect (e.g., a carbon balance). The carbon balance may be used to generate a token (when the carbon balance indicates a net environmental benefit) or consume a token (when the carbon balance indicates a net environmental detriment). The decarbonized products 404a may include one or more of methanol, ethanol, sustainable aviation fuel (SAF), acid, or any other suitable chemical, fuel, or compound. The value of the token may be based on the magnitude of one or more of the input greenhouse gases 402, the decarbonized products 404a, the output greenhouse gases 404b, the energy and utility input 406, the environmental effect, or the like.

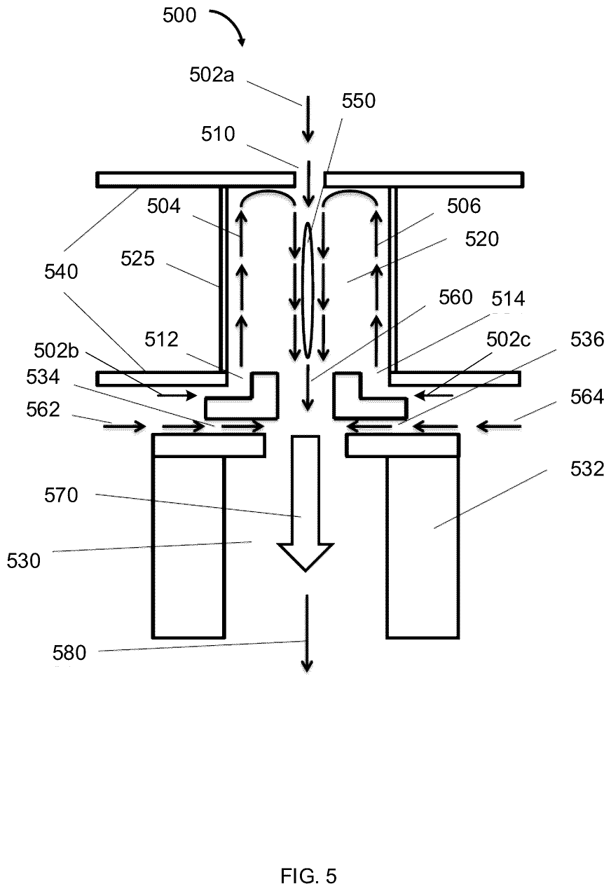

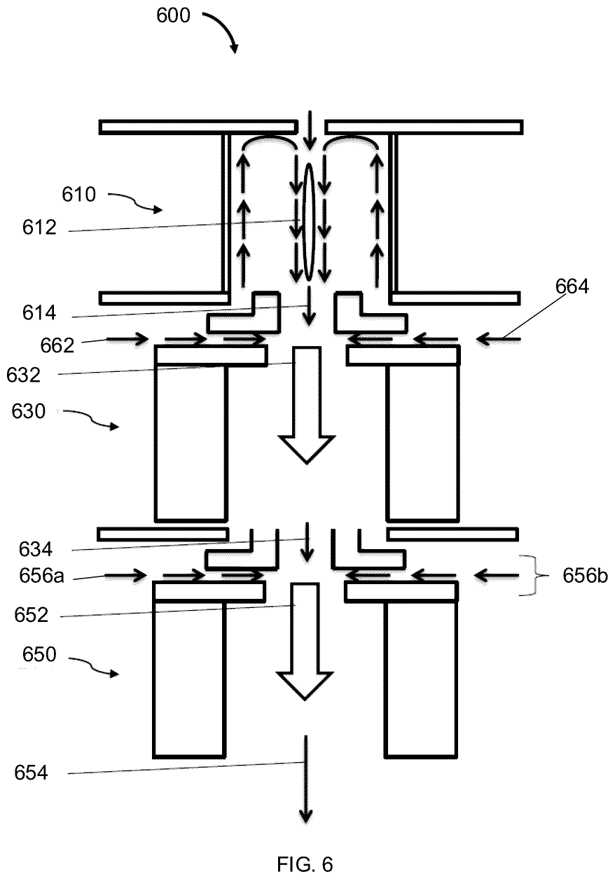

An example carbon conversion system may be a plasma reaction system 500, 600 (e.g., as illustrated in FIG. 5 or 6) which may allow for processing or reformation (or reforming, i.e., rearrangement of a molecular structure of hydrocarbons included in a gas) of gas by injecting unreacted gas after a plasma chamber (520 as illustrated in FIG. 5 or 610 as illustrated in FIG. 6) included in the plasma reaction system 500, 600. The amount of reformation may be tokenized and later sold, traded, invested, or the like.

In the example plasma reaction system 500, 600 (e.g., as illustrated in FIG. 5 or 6), any unreacted gas injected after the plasma chamber (520 as illustrated in FIG. 5 or 610 as illustrated in FIG. 6) may react with “waste” residual energy contained in the processed stream ((560 as illustrated in FIG. 5 or 614 as illustrated in FIG. 6) from the plasma chamber (520 as illustrated in FIG. 5 or 610 as illustrated in FIG. 6). This is accomplished with one or more inlets flows (562, 564 as illustrated in FIG. 5 or 662, 664 as illustrated in FIG. 6) designed to introduce the additional gas stream into the post-plasma chamber stream and effect mixing between the two gas streams. In cases where the reforming of the post-plasma stream is exothermic, the temperature of the mixed stream may be high enough for reforming to occur effectively. The unreacted gas (and all other inputs/input parameters) may be known, as may be the outputs/output parameters, so additional environmental benefits of the post-plasma chamber reactions may be tokenized.

FIG. 5 illustrates a cross-sectional view of a plasma reaction system 500 that includes a plasma chamber 520 and an ancillary reaction chamber 530. Sensors (not illustrated) may be positioned at any of the features described in conjunction with FIG. 5 to measure inputs, outputs, energy, utility, and/or parameters related to the plasma reaction system 500 and operation thereof. The plasma chamber 520 may include any number of inlets, including a first inlet 510, a second inlet 512, and a third inlet 514 through which one or more gases 502a, 502b, 502c may flow to enter the plasma chamber 520. Two or more of the first inlet 510, the second inlet 512, or the third inlet may be positioned on opposite or substantially opposite sides of the plasma chamber 520 such that a gas flow 502a corresponding to the first inlet 510 and a gas flow 502b corresponding to the second inlet 512 may generate forward and reverse vortex arrangements within the plasma chamber 520 that facilitate mixing and reaction of the gases 502a and 502b within the plasma chamber 520. The first inlet 510 and/or the second inlet 512 and/or the third inlet 514 may be positioned along any surface or other part of the plasma chamber 520 and oriented in any direction to facilitate the flow of the gases 502a, 502b, 502c into the plasma chamber 520 with different vortex arrangements between the first inlet 510 and/or the second inlet 512 and/or the third inlet 514. For example, the first inlet 510 may be positioned on a top surface of the plasma chamber 520 such that the gases 502a enter from the top of the plasma chamber 520, while the second inlet 512 and/or the third inlet 514 may be positioned on a bottom surface of the plasma chamber 520 such that the gases 502b, 502c enter from the bottom of the plasma chamber 520. As another example, the second inlet 512 and the third inlet 514 may both be along a lateral surface of the plasma chamber 520 with the second inlet 512 and the third inlet 514 being positioned opposite or substantially opposite with respect to each other. In some embodiments, the plasma chamber 520 may include one or more of the first inlet 510, or the second inlet 512, or the third inlet 514.

More than one inlet may be oriented in a particular direction such that the forward vortex arrangement (i.e., corresponding to the first inlet 510) and/or the reverse vortex arrangement (i.e., corresponding to the second inlet 512) may include a group of inlet ports. As illustrated in FIG. 5, for example, the reverse vortex arrangement may be formed by the gases 502b and 502c flowing through the second inlet 512 and/or a third inlet 514 that may be oriented in the same or a similar flow direction as the second inlet 512. Additionally or alternatively, the forward vortex arrangement may include more inlet ports than the first inlet 510, such as one or more inlet ports adjacent to the first inlet 510. The gases 502a, 502b, 502c entering the plasma chamber 520 via the group of inlet ports, which may contribute to the forward and/or the reverse vortex arrangements, may or may not mix together to form a single gas stream moving in the same direction. The gases 502b and 502c flowing through the second inlet 512 and the third inlet 514 may form gas flow streams 504 and 506, respectively, in which the gas flow streams 504, 506 enter the plasma chamber 520 as discrete streams that mix within the plasma chamber 520 after being redirected by the top surface of the plasma chamber 520.

One or more chamber walls 525 may enclose the plasma chamber 520 and demarcate an interior space of the plasma chamber 520 in which chemical reactions between gases flowing into the plasma chamber 520 may occur. The chamber walls 525 may be one or more of: opaque to gases, inert with respect to chemical reactions that occur within the plasma chamber 520, have a high melting temperature, or include a low coefficient of thermal expansion. For example, the chamber walls 525 may be include one or more of quartz, boron nitride, aluminum, ceramics, silicon carbide, tungsten, molybdenum, any other refractory materials, or a mixture thereof. Additionally or alternatively, the chamber walls 525 may be made of a radiofrequency-transparent material that allows energy directed by one or more waveguides 540 to feed a plasma 550 inside the plasma chamber 520. As such, energy from a microwave, electricity, or other source may be directed through the chamber walls 525 by the waveguides 540 to supply energy for the plasma 550 and the plasma chamber 520.

In these and other embodiments, an average temperature of the plasma chamber 520 may generally range from approximately 1,000 Kelvin (K) to approximately 3,500 K, while a peak temperature of the plasma 550 may reach approximately 50,000 K or higher. The temperature at particular locations within the plasma chamber 520 (e.g., in the center of the plasma chamber 520) may exceed the melting point of the chamber walls 525 and/or the waveguides 540 in some instances. Because the forward vortex arrangement and/or the reverse vortex arrangement of the gases 502a, 502b, 502c may provide an insulating effect, the chamber walls 525 and/or the waveguides 540 may not reach their respective melting points when the temperature at particular locations of the plasma chamber 520 exceeds those melting points.

The gases 502a, 502b, 502c in the plasma chamber 520 may include reactant gases involved in chemical reactions relating to natural gas reforming, syngas generation, reactant combustion, or any other chemical reactions that may be facilitated in a high-temperature reaction environment provided by the plasma chamber 520 in which heat from the plasma 550 may provide sufficient energy to break molecular bonds and/or initiate particular chemical reactions. An outlet gas stream 560 may include chemical products formed by the chemical reactions that occur in the plasma chamber 520 and unreacted reactants included in the gases 502a, 502b, 502c that entered the plasma chamber 520.

The outlet gas stream 560 may be mixed with one or more ancillary reaction chamber inlet flows 562, 564 to form an ancillary reaction chamber inlet flow 570. The ancillary reaction chamber inlet flows 562, 564 may include the same or similar gases as the gases 502a, 502b, 502c injected into the plasma chamber 520. Additionally or alternatively, the inlet flows 562, 564 may include reactants that were not present in the gases 502a, 502b, 502c and/or materials that facilitate the occurrence of one or more chemical reactions in the ancillary reaction chamber 530. For example, waste gases and/or liquids from related chemical processes or other plasma reactors may be included in the inlet flows 562, 564 to increase a waste-to-product reformation ratio of one or more of the waste gases or liquids. Further, waste-to-energy reformation may be increased relative to a threshold by including waste in the inlet flows 562, 564.

Oxidizer gases, such as air, oxygen, nitric oxide, etc., may be included in the inlet flows 562, 564 to drive particular chemical reactions and facilitate generation of particular chemical products. By including an ancillary reaction chamber 530 that obtains the outlet gas stream 560 and various other gases, a degree of reaction of one or more chemical reactants may be increased to increase the efficiency of the plasma reaction system 500. Additionally or alternatively, including the ancillary reaction chamber 530 in the plasma reaction system 500 may allow for a smaller plasma chamber 520 because the ancillary reaction chamber 530 may increase a conversion rate of chemical reactants. In these and other embodiments, the inlet flows 562, 564 may include a total flowrate ranging from approximately 50% up to approximately 5000% of the flowrate of the outlet gas stream 560 exiting the plasma chamber 520 to provide gases and/or liquids for chemical reactions to take place in the ancillary reaction chamber 530.

The inlet flows 562, 564 may be directed via one or more ancillary reaction chamber inlets 534, 536 to mix with the outlet gas stream 560 of the plasma chamber 520. In some embodiments, the ancillary reaction chamber inlets 534, 536 may be oriented at approximately 90o relative to the outlet gas stream 560 such that the inlet flows 562, 564 may be approximately perpendicular to the outlet gas stream 560. Additionally or alternatively, the ancillary reaction chamber inlets 534, 536 may be oriented at approximately at an angle ranging from approximately 30o to approximately 180o (i.e., countercurrent) relative to the outlet gas stream 560. Additionally or alternatively, a number of ancillary reaction chamber inlets and/or an orientation of each ancillary reaction chamber inlet may differ from the two ancillary reaction chamber inlets 534, 536 and the two inlet flows 562, 564 aimed at the same or similar orientations relative to the outlet gas stream 560. For example, a single ancillary reaction chamber inlet aimed at 180o relative to the outlet gas stream 560 may be used. As another example, three ancillary reaction chamber inlets aimed at varying angles relative to the outlet gas stream 560 may be used. A size and/or a number of ancillary reaction chamber inlets (e.g., two ancillary reaction chamber inlets 536, 536) may be set based on a selected flowrate through the plasma chamber 520 and/or the ancillary reaction chamber 530.

The ancillary reaction chamber inlet flow 570 may be directed towards the ancillary reaction chamber 530 for further processing of one or more of the gases included in the ancillary reaction chamber inlet flow 570. One or more walls 532 of the ancillary reaction chamber 530 may be made of a material that has a high thermal resistance and/or a low coefficient of thermal expansion. For example, the walls 532 may include one or more of carbon steel or other carbon composites, a nickel alloy, aerospace-grade aluminum, titanium, quartz, ceramics, tungsten, molybdenum, or any other suitable material, including any refractory materials.

The gases included in the ancillary reaction chamber inlet flow 570 may react in the ancillary reaction chamber 530 to yield one or more chemical products. The chemical products yielded by the chemical reactions in the ancillary reaction chamber 530 may include the same chemical products yielded by chemical reactions that occurred in the plasma chamber 520. Additionally or alternatively, the chemical products formed in the ancillary reaction chamber 530 may include various chemicals that are not formed in the plasma chamber 520 based on different chemical reactions facilitated by materials included in the inlet flows 562, 564 that were not present in the gases 502a, 502b, 502c that entered the plasma chamber 520.

After mixing, an ancillary reaction chamber 530 may provide sufficient residence time for reformation to occur in the mixed-gas stream. Additionally or alternatively, the ancillary reaction chamber 530 may be recuperatively or externally cooled. The gas stream 580 may leave the ancillary reaction chamber 530 and flow into piping or tubing for further processing or storage of the gases. Additionally or alternatively, the plasma chamber 520 and the ancillary reaction chamber 530 may be a single unit such that the formation of the syngas occurs in the plasma-chamber-ancillary-reaction-chamber-combined unit.

In these and other embodiments, the chemical reactions occurring in the ancillary reaction chamber 530 may be facilitated by heat carried over from the plasma chamber 520. As such, the ancillary reaction chamber 530 may not include any plasma, and energy sources for heating the plasma 550 may not be directed towards the ancillary reaction chamber 530. The absence of plasma and/or directed energy sources may cause the ancillary reaction chamber 530 to operate at lower temperatures than the plasma chamber 520, and the ancillary reaction chamber 530 may include a larger volume and/or operate at a same or different pressure (e.g., higher or lower) than the plasma chamber 520 to facilitate the occurrence of the chemical reactions. Additionally or alternatively, the ancillary reaction chamber 530 may be made of a less heat-resistant material than the plasma chamber 520 because the ancillary reaction chamber 530 may operate at lower temperatures than the plasma chamber 520. For example, the plasma chamber 520 may include an aerospace grade aluminum, while the ancillary reaction chamber 530 may include a molybdenum metal.

The chemical products formed during chemical reactions occurring in the ancillary reaction chamber 530, any unreacted chemical reactants, and any other gases included in the ancillary reaction chamber 530 may be directed out of the ancillary reaction chamber 530 in an outlet gas flow 580. The outlet gas flow 580 may be sent to an ancillary equipment of the plasma reaction system 500, such as a scrubber, a pressure-swing adsorption unit, an amine unit, and/or a compressor. Additionally or alternatively, the outlet gas flow 580 may be sent to a second-stage ancillary reaction chamber for further processing of the products, unreacted chemicals, and/or any other gases included in the outlet gas flow 580. Additionally or alternatively, the outlet gas flow 580 may be sent to an integrated reformer for further processing of the products, unreacted chemicals, and/or any other gases included in the outlet gas flow 580.

As illustrated in FIG. 6, a plasma reaction system 600 may include: (i) a plasma chamber 610 in fluid communication with (ii) an ancillary reaction chamber 630; and (iii) an integrated reformer 650 in fluid communication with the ancillary reaction chamber. Sensors (not illustrated) may be positioned at any of the features described in conjunction with FIG. 6 to measure inputs, outputs, energy, utility, and/or parameters related to the plasma reaction system 600 and operation thereof. The ancillary reaction chamber 630 may be configured to use heat from a heated first synthesis gas stream 614 received from the plasma chamber to initiate an exothermic reaction with one or more second gas streams 662, 664 to create a reaction product 632 which may be output as a heated second synthesis gas stream 634 to the integrated reformer 650. The “heated second synthesis gas stream” may be the reaction product of the reaction between the heated first synthesis gas stream and the second gas stream.

The plasma reaction system 600 may include a plasma chamber 610 connected with one or more ancillary reaction chambers (e.g., ancillary reaction chamber 630), and one or more integrated reformers (e.g., integrated reformer 650) in series. The plasma chamber 610 may be the same as or similar to the plasma chamber 520 as described in relation to FIG. 5. As such, the plasma chamber 610 may be configured to obtain one or more inlet flows in which each inlet flow may include one or more gases and/or a particular vortex arrangement. Additionally or alternatively, the plasma chamber 610 may include a plasma that is heated by an energy source, such as a microwave source or an electricity source. In these and other embodiments, the first ancillary reaction chamber 630 may be the same as or similar to the ancillary reaction chamber 530 as described in relation to FIG. 5. As such, the ancillary reaction chamber 630 may have a size or volume greater than a size or volume of the plasma chamber 610 and/or may operate at a pressure the same as or different than a pressure of the plasma chamber 610.

Also after the plasma chamber 610 and/or the ancillary reaction chamber 630, an integrated reformer 650 may be present. The integrated reformer 650 may be a discrete reaction unit that may be connected to the plasma chamber 610 and/or to the ancillary reaction chamber 630. Additionally or alternatively, the plasma chamber 610 and the integrated reformer 650 may be a single unit such that the formation of the syngas by the integrated reformer 650 occurs in the plasma-chamber-integrated-reformer combined unit.

The integrated reformer 650 may be connected to the ancillary reaction chamber 630 by having an outlet flow of a heated second synthesis gas stream 634 of the first ancillary reaction chamber 630 mixed with one or more third gas stream 656a, 656b (e.g., an input flow into the integrated reformer 650) and feed into the integrated reformer 650 as an integrated reformer inlet flow 652.

The integrated reformer 650 may not be connected to a heat source, such as a plasma 612 used to heat the plasma chamber 610. The integrated reformer may use heat from a heated second synthesis gas stream 634 to initiate an endothermic reaction to generate outlet flow 654 (e.g., gas product, such as syngas). As such, chemical reactions that may occur in the integrated reformer 650 between gases included in the integrated reformer flow 652 may be facilitated by heat from the ancillary reaction chamber 630, which may be received by the integrated reformer 650 along with the gases in the outlet flow of a heated second synthesis gas stream 634 of the ancillary reaction chamber 630.

A temperature of the integrated reformer 650 may be less than the temperature of the ancillary reaction chamber 630. As such, the integrated reformer 650 may be made of a material that is less heat resistive and/or include a greater coefficient of thermal expansion than a material used for the ancillary reaction chamber 630 and/or the plasma chamber 610. Additionally or alternatively, the integrated reformer 650 may include a greater volume and/or operate at a same or different pressure than the ancillary reaction chamber 630 to facilitate chemical reactions that occur in the integrated reformer 650.

An outlet flow 654 of the integrated reformer 650 may be sent to an ancillary equipment of plasma reaction system, such as a scrubber, a pressure-swing adsorption unit, an amine unit, and/or a compressor, for further processing of the gases included in the outlet flow 654. The outlet flow 654 may be directed towards one or more additional ancillary reaction chambers and/or integrated reformers, such as a second ancillary reaction chamber in series, a second and a third ancillary reaction chamber in series, etc. or any combination. In these and other embodiments, an operating temperature of each subsequent ancillary reaction chamber in the series of ancillary reaction chambers and/or integrated reformers may be less than the operating temperature of the previous chamber in the series. As such, each subsequent chamber may have a greater size and/or volume and/or a same or different pressure than the previous chamber in the series.

The outlet flow 654 of the integrated reformer 650, the outlet flow of a heated second synthesis gas stream 634 of the ancillary reaction chamber 630, and/or an outlet flow of a heated first synthesis gas stream 614 of the plasma chamber 610 may be directed towards one or more chambers (e.g., ancillary reaction chambers, integrated reformers) that may be configured in parallel with respect to one another.

For systems that include a plasma chamber 610, an ancillary reaction chamber 630, and an integrated reformer 650, each of these units may be individual units, or may be combined in a plasma-chamber-ancillary-reaction-chamber-integrated-reformer combined unit, in which the ancillary reaction chamber 630 and the integrated reformer 650 may be coupled to the plasma chamber 610, and to each other, in any order, combination, including in series, parallel, or a combination thereof. For systems that include a plasma chamber 610, an ancillary reaction chamber 630, and an integrated reformer 650, the work done by each of these units may be used to generate tokens. In some embodiments, the net or aggregate environmental benefits may be used to tokenize. Alternatively, less than all of the components may be used to adjust the tokens.

When system (e.g., system 400 as illustrated in FIG. 4) reduces or reclaims carbon, techniques may be employed to verify, validate, and/or prove that the system has performed the work, or at least that the output(s) of the system have the effect of carbon reduction and/or reclamation. These techniques may be used on existing systems or to other systems and may verify, validate, and/or prove using any criteria, variables, or factors, including input unit volume, time elapsed between input and output, system uptime, location of the system, temperatures of components of the system, or the like.

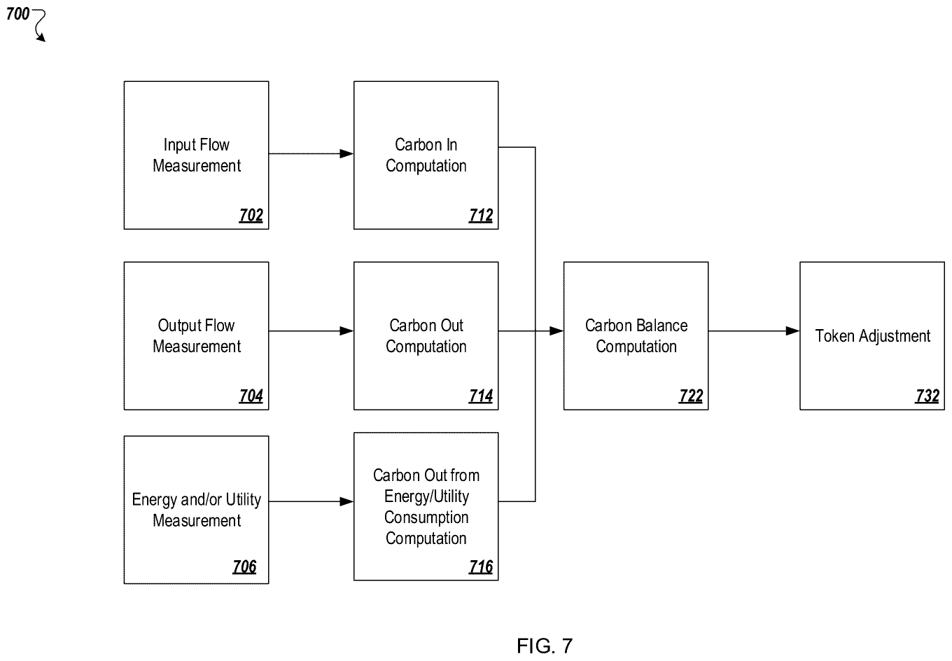

FIG. 7 illustrates an example process flow 700 for token adjustment (e.g., token generation or consumption) that may be used for any system (e.g., for a system 100, 300, 400 as illustrated in FIGS. 1, 3, and 4, respectively). Some references may be made to the systems 300, 400 of FIGS. 3 and 4 for ease in explanation, but any system may be implemented. FIG. 7 may also be described in the context of a carbon token to associate with or attach to a decarbonized product, but any input, process, system, and/or output may be used.

The process flow 700 may include one or more measurements of input(s) to a system, including an input flow measurement 702. The input flow measurement may be received from an input flow sensor. The inputs may include any input described herein including flowrate, composition, temperature, pressure, materials, or the like. In an example and in the context of a carbon to decarbonized product, a “carbon in” computation 712 may be performed to measure a total amount of carbon that is provided to the system. In an example, the carbon input may be calculated by taking a total input flowrate multiplied by a carbon species composition.

The process flow 700 may include taking one or more measurements of output(s) from the system, including an output flow measurement 704. The output flow measurement may be received from an output flow sensor. The outputs may include any output described herein including flowrate, composition, temperature, pressure, materials, or the like. In an example and in the context of the carbon to decarbonized product, a “carbon out” computation 714 may be performed to measure a total amount of carbon that is output by the system. In an example, the “carbon out” may be calculated by taking a total output flowrate multiplied by a carbon species composition.

The process flow may also include taking one or more energy and/or utility measurements 706, including a measurement of electricity, fuel, or any other suitable utilities. The energy and/or utility measurements may be received from an energy and/or utility sensor. In an example and in the context of the carbon to decarbonized product, an energy/utility consumption computation 716 may be performed to measure a total amount of energy/utility used by the system for decarbonization. The carbon out for the energy/utility consumption may be calculated by taking the emissions factors multiplied by the energy or utility.

The process flow 700 may be used to compute an amount of work performed by the system. In an example, the amount of work performed may be characterized by a carbon balance computation 722 (e.g., which may be carbon reduction calculation). The carbon reduction calculation may be determined based on a net difference between a “carbon in 712” versus a “carbon out 714” and carbon out from energy/utility 716.

As illustrated, the carbon reduction calculation may be performed using the “carbon in” calculation, minus the “carbon out” calculation, the net difference being indicative of an amount of carbon reduction that the system has achieved. That is, when the difference between the “carbon in” computation and the “carbon out” computation is positive, then a net amount of carbon reduction has been achieved. When the difference between the “carbon out” computation and the carbon in” computation is positive, then a net amount of carbon increase (i.e., carbon emission) has been achieved.

In some embodiments, the carbon reduction calculation may account for any other factor, including an amount of energy or utilities used to achieve the carbon reduction. That is, the carbon reduction calculation may include the amount of the carbon out from energy or utilities used to achieve carbon reduction. The modified carbon reduction calculation may be: the “carbon in” computation minus the “carbon out” computation minus the “carbon out” from the amount of energy or utility used to achieve carbon reduction. When the difference is positive, then a net amount of carbon reduction has been achieved with respect to these factors (i.e., carbon in, carbon out, energy/utility used). When the difference is negative, then a net amount of carbon increase has occurred with respect to these factors (i.e., carbon in, carbon out, energy/utility used).

A carbon balance computation 722 may be performed based on the one or more measurements (e.g., input flow measurement, output flow measurement, energy measurement, utility measurement, or any other suitable measurements such as composition, temperature, pressure, materials, or the like). When the carbon balance is positive and carbon has been increased, then a token may be consumed. When the carbon balance is negative and carbon had been reduced, then a token may be generated. The amount of carbon increase or carbon reduction may be used to consume or generate the token. That is, when the amount of carbon reduction is greater in magnitude compared to a single unit (e.g., 5 units instead of 1 unit), then the token value may be correlated to the magnitude of the carbon reduction (e.g., generating a token having a value of 5 instead of generating a token having a value of 1). When the amount of carbon increase is greater in magnitude compared to a single unit (e.g., 5 units of carbon increase instead of 1 unit of carbon increase) then the token value may be correlated to the magnitude of carbon increase (e.g., consuming a token having a value of 5 instead of consuming a token having a value of 1). Based on the carbon balance, a token may be generated (e.g., for carbon reduction) or consumed (e.g., for carbon increase). In addition or alternatively, the token may be validated using one or more factors (e.g., location data).

One or more sensors may measure the inputs (e.g., carbon) to the system (e.g., system 400 as illustrated in FIG. 4), measure the outputs, and measure energy and/or utility and may determine between the inputs and outputs, a reduction, reclamation, or repurpose of carbon. The input may be measured in a unit volume and/or mass and so for a given unit of volume and/or mass (and type of input) and for a certain product or output type, one may expect to have a certain carbon balance. By measuring the product or output and energy or utility and comparing to the input, one may validate the work done by the system. Other variations may be used for validation, including specific validation metrics for different types of inputs, products, outputs, energy, utility, or for any type of processing within the system.

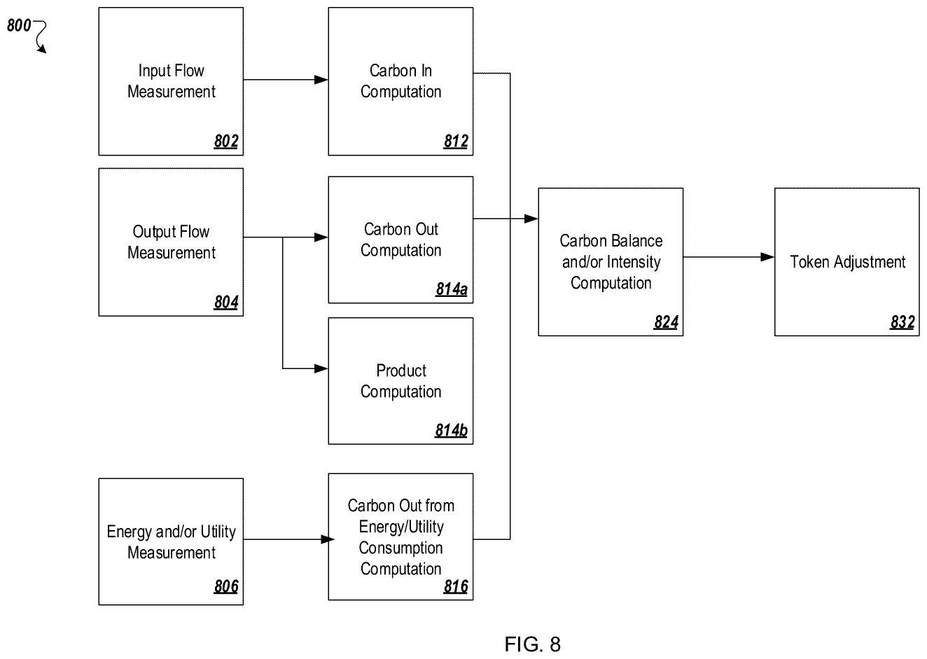

FIG. 8 illustrates an example process flow 800 for token generation that may be used for a system (e.g., system 100, 300, 400 as illustrated in FIGS. 1, 3, and 4, respectively). Some references may be made to the systems of FIGS. 3, 4 and 7 for ease in explanation, but any system may be implemented. FIG. 8 may also be described in the context of a carbon to decarbonized product, but any input, process, system, and output may be used.

FIG. 8 may include some of the features of FIG. 7. That is, the input flow measurement 802, the output flow measurement 804, the energy and/or utility measurement 806, the carbon in computation 812, the carbon out computation 814a, and the carbon out from the energy/utility consumption computation 816 may include the functionality disclosed with respect to FIG. 7 for the input flow measurement 702, the output flow measurement 704, the energy and/or utility measurement 706, the carbon in computation 712, the carbon out computation 714, and the carbon out from the energy/utility consumption computation 716.

The process flow 800 of FIG. 8 may include a different output measurement, which may account for a product quantity. A product computation 814b may be performed, which may be based on the total flowrate and product composition. The amount of work performed may be calculated based on the product computation 814b, and may be represented as a per product feature. That is, the carbon balance computation may be a carbon intensity (or carbon reduction density) computation which may be calculated as: the “carbon in” computation 812 minus the “carbon out” computation 814a minus the carbon out amount from energy or utility used to achieve carbon reduction (e.g., the carbon out from the energy/utility consumption computation 816), with the result being divided by the product computation 814b. This carbon intensity computation 824 allows a token to be generated or consumed on a per product basis, rather than on an aggregate basis. In addition or alternatively, the token may be validated using one or more factors (e.g., location data).

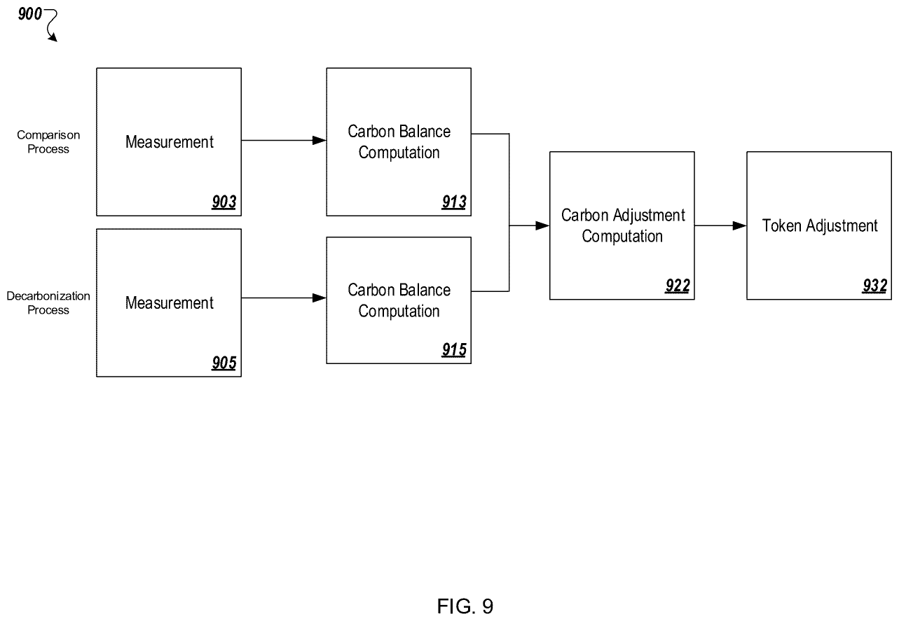

FIG. 9 illustrates an example process flow 900 for token adjustment (e.g., token generation or token consumption) that may be used for a system (e.g., system 100, 300, 400 as illustrated in FIGS. 1, 3, and 4, respectively). Some references may be made to the systems 300, 400 of FIGS. 3 and 4 for ease in explanation, but any system may be implemented. FIG. 9 may be described in the context of a carbon to decarbonized product, but any input, process, system, and output may be used.

FIG. 9 further describes token adjustment (e.g., token generation or token consumption) based on a comparison between two processes. For example, there exists a first process (e.g., a comparison process) and a technique for generating a token may be based on a comparison between a second process (e.g., decarbonization process) and a first process. One or more measurements 903, 905 may be performed for the first process (e.g., a comparison process) and the second process (e.g., decarbonization process), respectively. One or more carbon balance computations 913, 915 may be performed for the first process (e.g., a comparison process) and the second process (e.g., decarbonization process), respectively. A carbon adjustment computation 922 may be performed to compare the first process (e.g., a comparison process) and the second process (e.g., decarbonization process). The carbon adjustment computation 922 may be used to facilitate a token adjustment 932. A token may be generated (e.g., when the second process results in increased carbon reduction when compared to the first process) or consumed (e.g., when the first process results in increased carbon reduction when compared to the second process). The value of the difference in carbon reduction balances may be used to generate or consume the token having a specific value. In addition or alternatively, the token may be validated using one or more factors (e.g., location data).

Any of the inputs, outputs, energy, utility, criteria, variables, or factors, or other calculations may be written to a distributed ledger based on block chain or other technologies, which may add the benefit of securing the results and actual performance of the disclosed systems. In these systems, there may be a secured, and tamper-resistant system that takes various measurements in the system and then writes to the block chain. If the measurement and/or block chain write system is tampered with, the related items in the ledger may be invalidated, which may serve as deterrent to tamper with the results and block chain write. Instead of invalidating items in the ledger, there may be a delay in writing to the block chain so that work would not need to be undone in the event of data and/or measurement tampering.

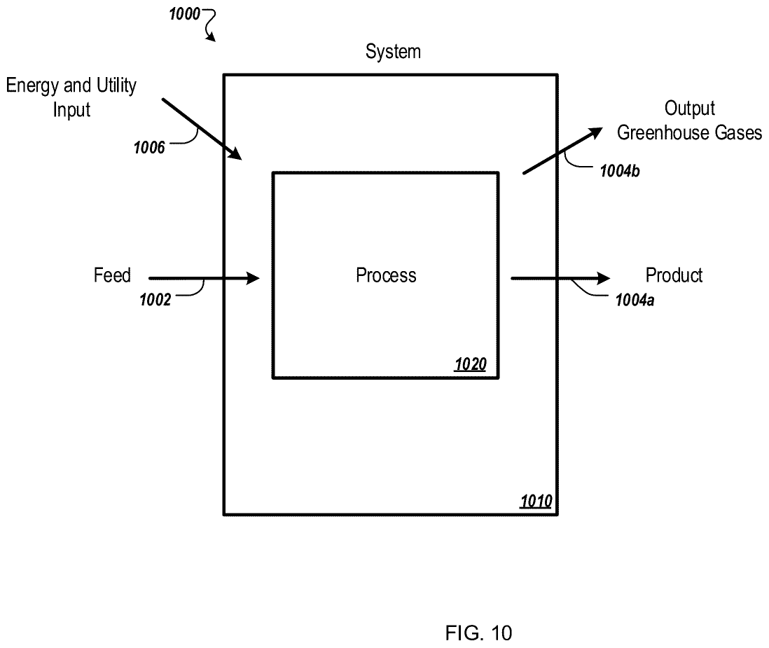

FIG. 10 illustrates a token adjustment process 1000 that includes a system 1010 that may include a process 1020 (e.g., a decarbonization process) that is based on a feed 1002, an output including a product 1004a and output greenhouse gases 1004b, and an energy & utility input 1006. The feed 1002 may include an amount of carbon and the product 1004a may include a different amount of carbon. The token may be generated or consumed based on a difference between the feed and the output, in which the difference may represent an environmental benefit, such as a carbon reduction. Alternatively or in addition, the token may be generated or consumed based on the energy & utility input 1006. That is, energy and utility input 1006 (which may include an emissions from power generation and utility consumption) may be a factor in determining carbon output. A result of the token adjustment process may be stored in a record or ledger, such as a blockchain. Any token generation may be recorded in a blockchain and any token consumption may be recorded in a blockchain. The token adjustment 732, 832, 932, as illustrated in FIGS. 7, 8, and 9, respectively, may be similar to the token adjustment 232 and may also be used in conjunction with the token adjustment process 1000.

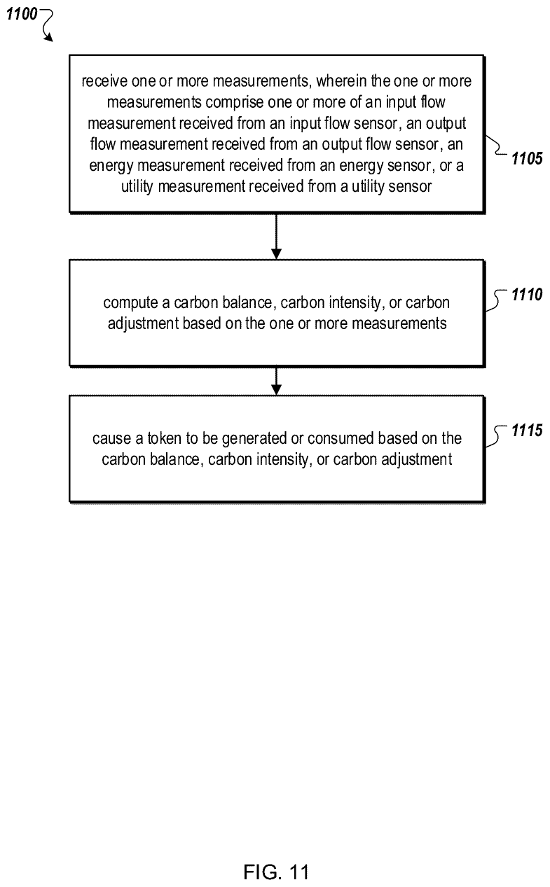

FIG. 11 is a flowchart of an example arrangement of operations for a method 1100 of token adjustment. The method 1100 may be performed by processing logic that may include hardware (circuitry, dedicated logic, etc.), software (such as is run on a general purpose computer system or a dedicated machine), or a combination of both, which processing logic may be included in any computer system or device. The software may be instructions or code capable of running on a virtualization environment and/or containerization environment such as bytecode and containerized program.

The method 1100, at operation 1105, includes receiving one or more measurements, where the one or more measurements include one or more of an input flow measurement received from an input flow sensor, an output flow measurement received from an output flow sensor, an energy measurement received from an energy sensor, or a utility measurement received from a utility sensor.

At operation 1110, the method 1100 may include computing a carbon balance, carbon intensity, or carbon adjustment based on the one or more measurements.

At operation 1115, the method 1100 may include causing a token to be generated or consumed based on the carbon balance, carbon intensity, or carbon adjustment.

Modifications, additions, or omissions may be made to the method 1100 without departing from the scope of the present disclosure. For example, in some examples, the method 1100 may include any number of other components that may not be explicitly illustrated or described.

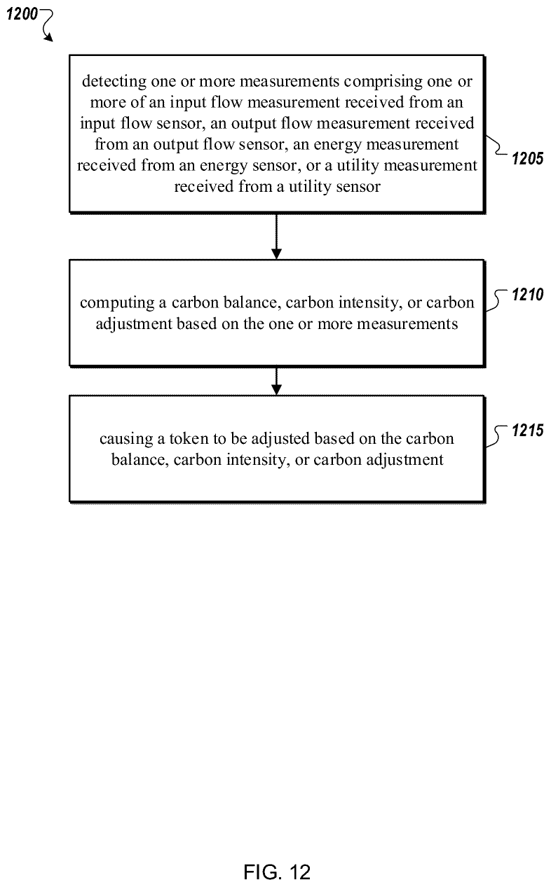

FIG. 12 is a flowchart of an example arrangement of operations for a method 1200 for adjusting tokens. The method 1200 may be performed by processing logic that may include hardware (circuitry, dedicated logic, etc.), software (such as is run on a general purpose computer system or a dedicated machine), or a combination of both, which processing logic may be included in any computer system or device. The software may be instructions or code capable of running on a virtualization environment and/or containerization environment such as bytecode and containerized program.

The method 1200, at operation 1205, includes detecting one or more measurements including one or more of an input flow measurement received from an input flow sensor, an output flow measurement received from an output flow sensor, an energy measurement received from an energy sensor, or a utility measurement received from a utility sensor.

At operation 1210, the method 1200 may include computing a carbon balance, carbon intensity, or carbon adjustment based on the one or more measurements.

At operation 1215, the method 1200 may include causing a token to be adjusted based on the carbon balance, carbon intensity, or carbon adjustment.

Modifications, additions, or omissions may be made to the method 1200 without departing from the scope of the present disclosure. For example, in some examples, the method 1200 may include any number of other components that may not be explicitly illustrated or described.

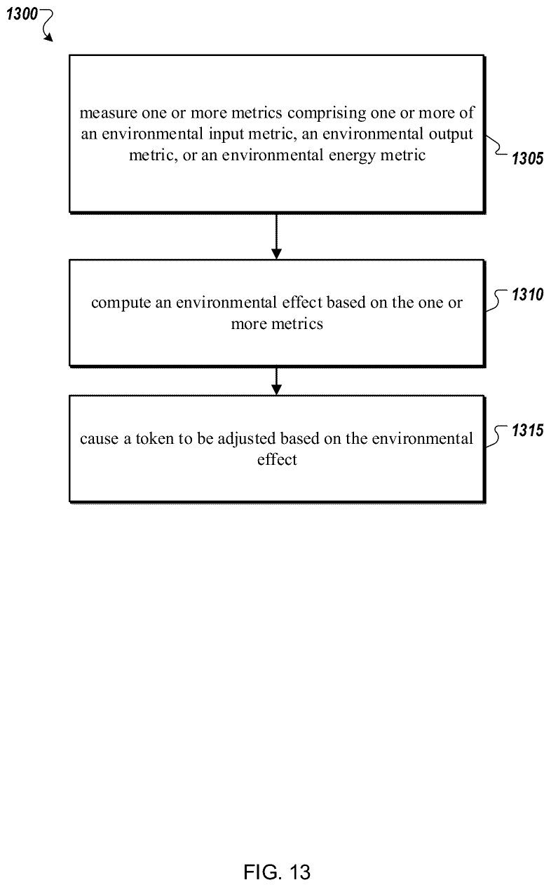

FIG. 13 is a flowchart of an example arrangement of operations for a method 1300 for adjusting tokens. The method 1300 may be performed by processing logic that may include hardware (circuitry, dedicated logic, etc.), software (such as is run on a general purpose computer system or a dedicated machine), or a combination of both, which processing logic may be included in any computer system or device. The software may be instructions or code capable of running on a virtualization environment and/or containerization environment such as bytecode and containerized program.

The method 1300, at operation 1305, includes measuring one or more metrics including one or more of an environmental input metric, an environmental output metric, or an environmental energy metric.

At operation 1310, the method 1300 may include computing an environmental effect based on the one or more metrics.

At operation 1315, the method 1300 may include causing a token to be adjusted based on the environmental effect.

Modifications, additions, or omissions may be made to the method 1300 without departing from the scope of the present disclosure. For example, in some examples, the method 1300 may include any number of other components that may not be explicitly illustrated or described.

For simplicity of explanation, methods and/or process flows described herein are depicted and described as a series of acts. However, acts in accordance with this disclosure may occur in various orders and/or concurrently, and with other acts not presented and described herein. Further, not all illustrated acts may be used to implement the methods in accordance with the disclosed subject matter. In addition, those skilled in the art will understand and appreciate that the methods may alternatively be represented as a series of interrelated states via a state diagram or events. Additionally, the methods disclosed in this specification are capable of being stored on an article of manufacture, such as a non-transitory computer-readable medium, to facilitate transporting and transferring such methods to computing devices. The term article of manufacture, as used herein, is intended to encompass a computer program accessible from any computer-readable device or storage media. Although illustrated as discrete blocks, various blocks may be divided into additional blocks, combined into fewer blocks, or eliminated, depending on the desired implementation.

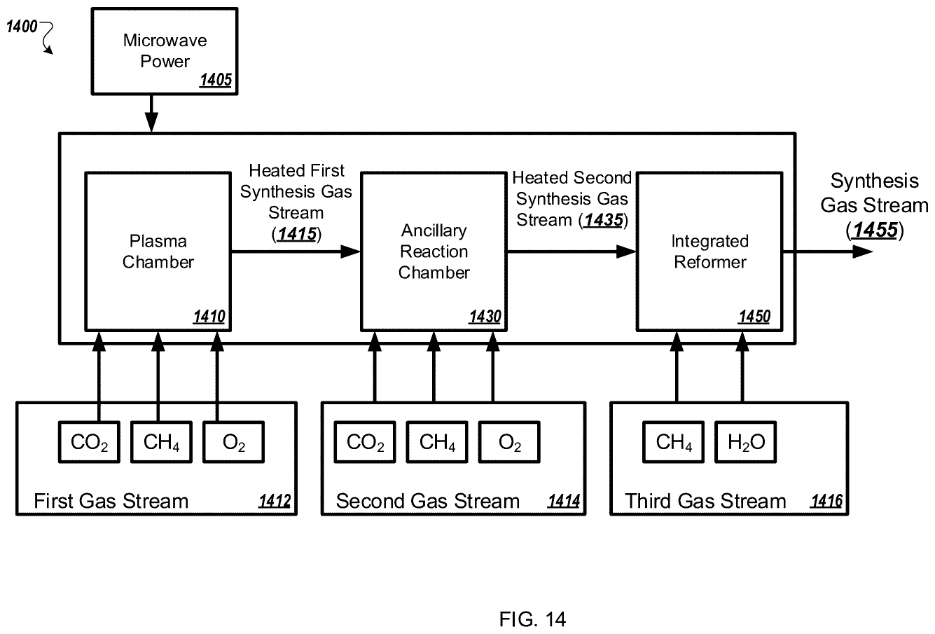

As illustrated in FIG. 14, synergies between components or beneficial results of a combination of components may be tokenized as shown in the system 1400. The system 1400 may include a plasma chamber 1410 that may receive microwave power 1405 and a first gas stream 1412 which may include one or more of CO₂, CH₄, O₂, or the like. The microwave power may cause an exothermic reaction to generate a heated first synthesis gas stream. When using the high thermal energy of the product gas from plasma chamber 1410 and ancillary reaction chamber 1430 as a result of the exothermic reaction, no extra heat or energy may be used for the further reforming reactions in the integrated reformer 1450. Also, using the steam produced from the plasma chamber 1410 and the ancillary reaction chamber 1430 for steam feed may use less external steam feed and less energy than a comparison baseline steam reforming process. The difference between the process 1400 using the steam and heat produced from the plasma chamber 1410 and the ancillary reaction chamber 1430 and the comparison baseline process may be tokenized as described with respect to FIG. 9. The resulting synthesis gas stream (e.g., syngas product) of the present system may have a ratio H₂:CO of from about 1:1 to about 3:1.

The heated first synthesis gas stream 1415 output from the plasma chamber 1410 may be input to the ancillary reaction chamber 1430. The ancillary reaction chamber may be operable to receive a second gas stream 1414. The second gas stream may include one or more of CO₂, CH₄, O₂, or the like. The second gas stream may include the same components as the first gas stream. The heated first synthesis gas stream 1415 and the second gas stream may react to generate a heated second synthesis gas stream 1435 which may be input to the integrated reformed. The integrated reformer 1450 may receive a third gas stream 1416 including one or more of CH₄ or H₂O. The natural gas received by the integrated reformed may react with H₂O in a reforming reaction to generate a synthesis gas stream 1455.

The reaction in the ancillary reaction chamber 1430 uses the thermal energy from the heated first synthesis gas stream 1415. The third gas stream 1416 to the integrated reformer 1450 may include any feed, and, as illustrated, may be natural gas (CH₄) and water (H₂O) (which may be in the form of steam or in the form of water used to generate steam inside the integrated reformer 1450). The integrated reformer 1450 may use the heat from the heated second synthesis gas stream 1435 without an external heat input. The amount of external heat saved by using the heat from one or more of the heated first synthesis gas stream 1415 or the second synthesis gas stream 1435 to drive the reaction may be used to generate a token. Alternatively or in addition, the amount of external heat used (when heat from one or more of the heated first synthesis gas stream 1415 or the second synthesis gas stream 1435 is not used to drive the reaction, e.g., for a comparison process that may lack this feature) may be used to consume a token. Alternatively or in addition, the amount of external heat used in addition to the heat from one or more of the heated first synthesis gas stream 1415 or the second synthesis gas stream 1435 to drive the reaction (e.g., for a comparison process that may lack this feature) may be used to consume a token.

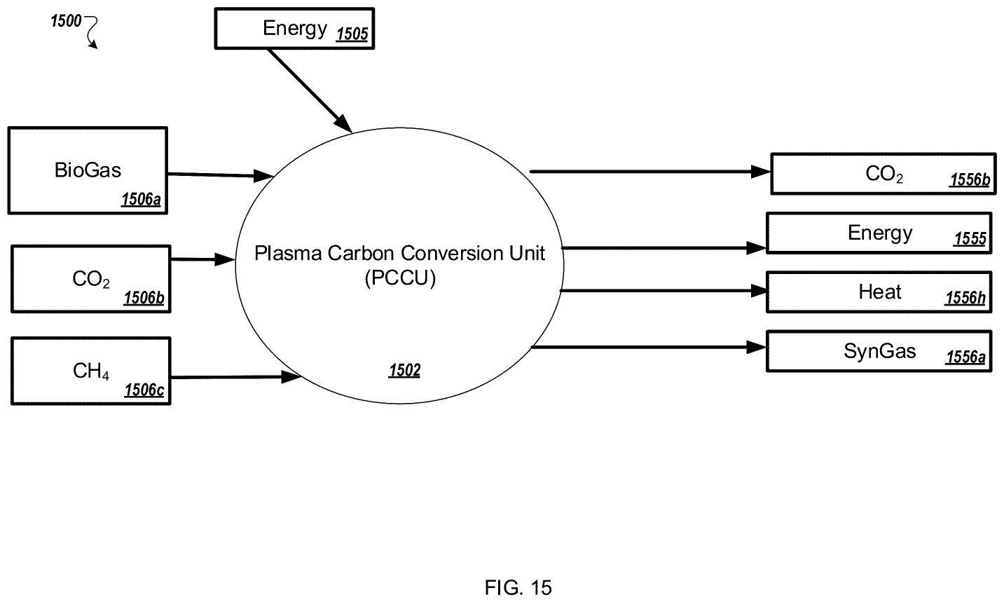

As illustrated in FIG. 15, a plasma reaction system 1500 may include a plasma carbon conversion unit 1502 (PCCU) that may facilitate a high temperature reaction environment to drive chemical reactions to completion. Sensors (not illustrated) may be positioned at any of the features described in conjunction with FIG. 15 to measure inputs, outputs, energy, utility, and/or parameters related to the plasma reaction system 1500 and operation thereof. Chemical reactions for dissociating carbon dioxide 1506b and/or generating various product gases, such as hydrogen gas and carbon monoxide, may be performed using the PCCU 1502. The chemical reactions performed using the PCCU 1502 may provide increased yields of the product gases relative to baseline amounts (which may not use the PCCU 1502) because the PCCU 1502 may facilitate energy for driving the formation of the product gases. Additionally or alternatively, the PCCU 1502 may include unreacted (or remaining) carbon dioxide gas 1556b in addition to converting the biogas 1506a into synthesis gas (syngas 1556a). Energy generated by the PCCU 1502 may drive further operations of other processing units and/or chemical reactions. For example, excess heat from the plasma reactor (e.g., plasma chamber 610 as illustrated in FIG. 6) may be used to drive operations of integrated reformers 650, heat utilization units, compressors, or any other processes of the formation, recycling, and/or separation of various product gases.

The PCCU 1502 may use various inputs including one or more of biogas 1506a, carbon dioxide 1506b, methane gas 1506c (or any other hydrocarbon gases), and input energy to yield various outputs including one or more of carbon dioxide gas 1556b, energy 1555, heat 1556h, syngas 1556a, steam, or the like. The amount of carbon dioxide 1506b included in the input stream to the PCCU 1502 may be greater than the amount of carbon dioxide 1556b included in the output stream of the PCCU 1502. Additionally, the input energy 1505 may be less than the output energy 1555 yielded by the PCCU 1502 because the chemical reactions occurring in the PCCU 1502 may be exothermic reactions that may generate heat 1556h. The energy 1555 generated by the chemical reactions and/or the unreacted carbon dioxide 1556b from the chemical reactions occurring in the PCCU 1502 may be recycled and included in as inputs to the PCCU 1502 to facilitate further chemical reactions or dissociation of carbon dioxide.

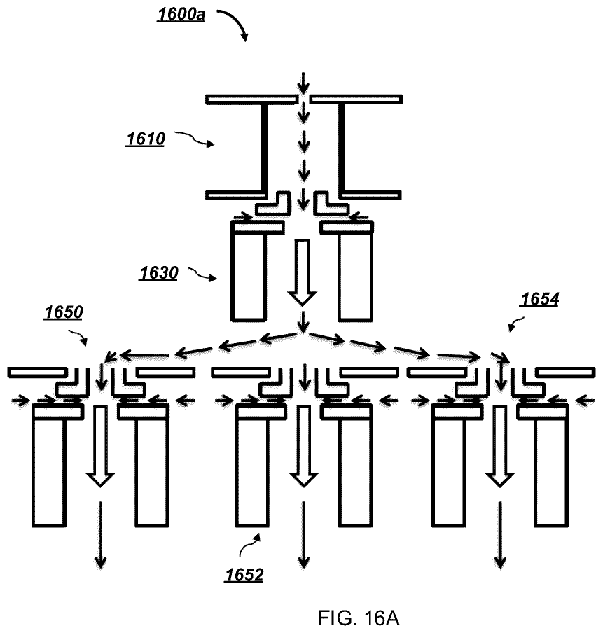

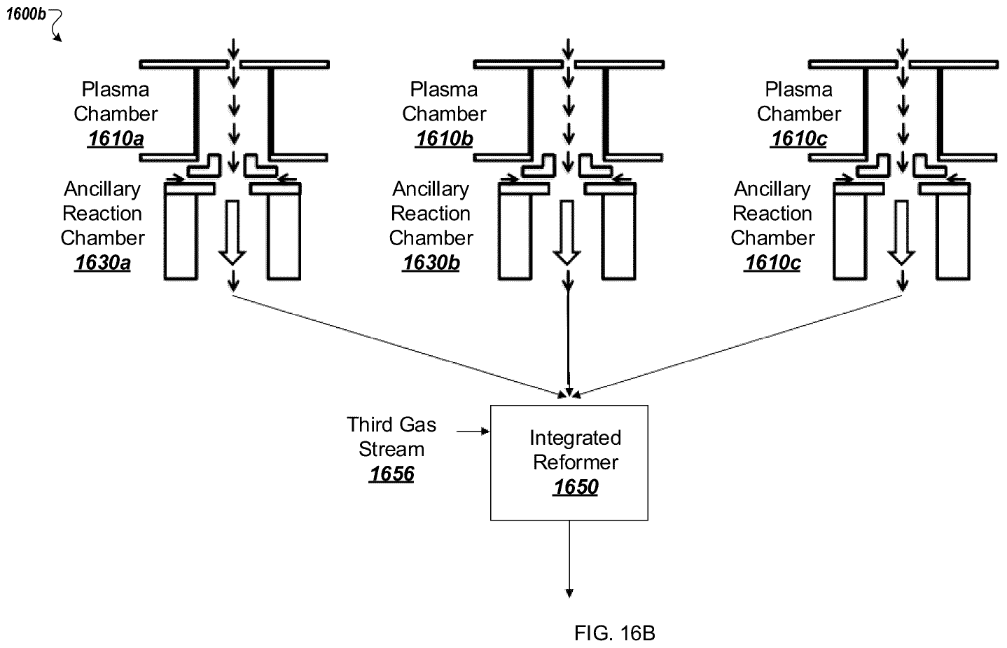

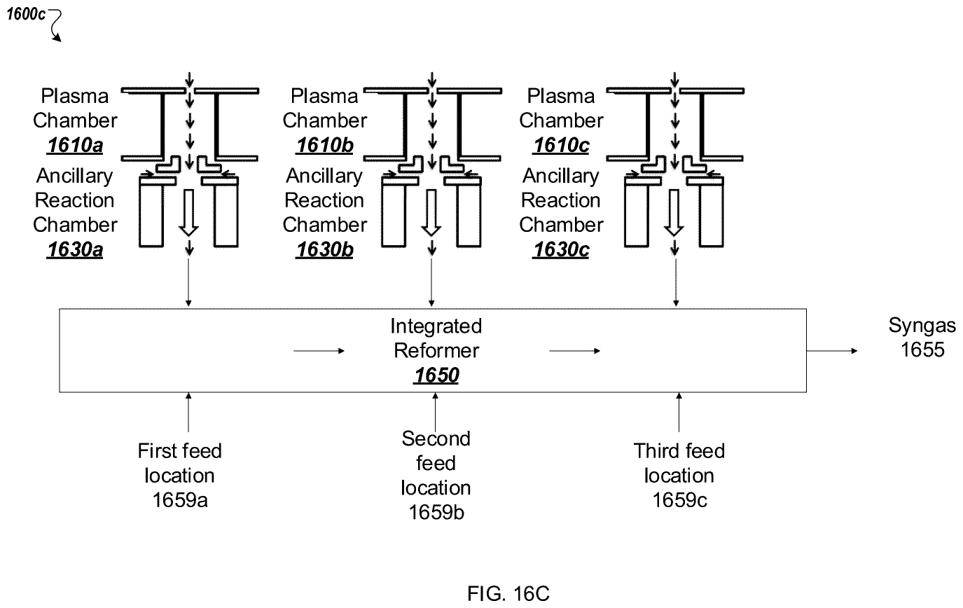

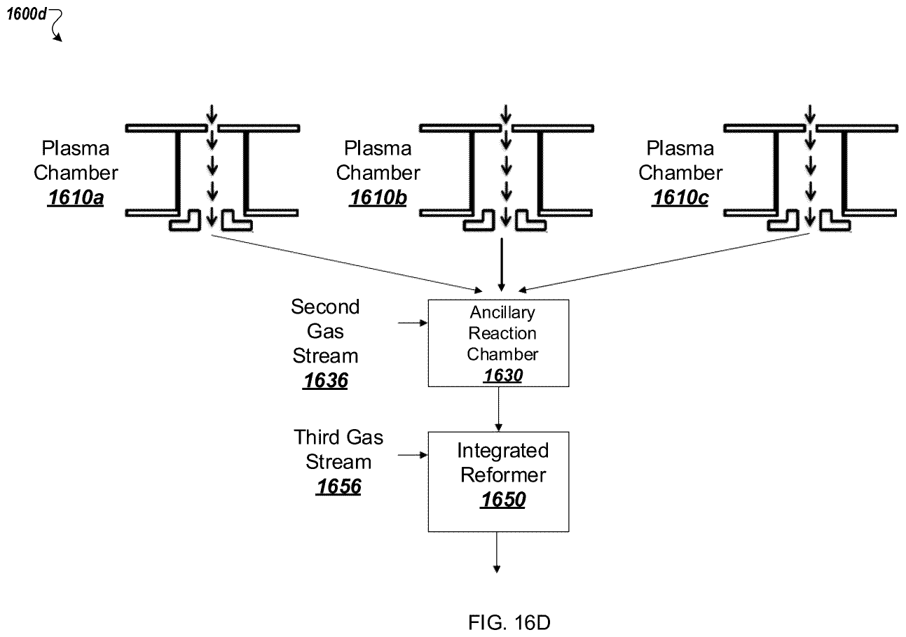

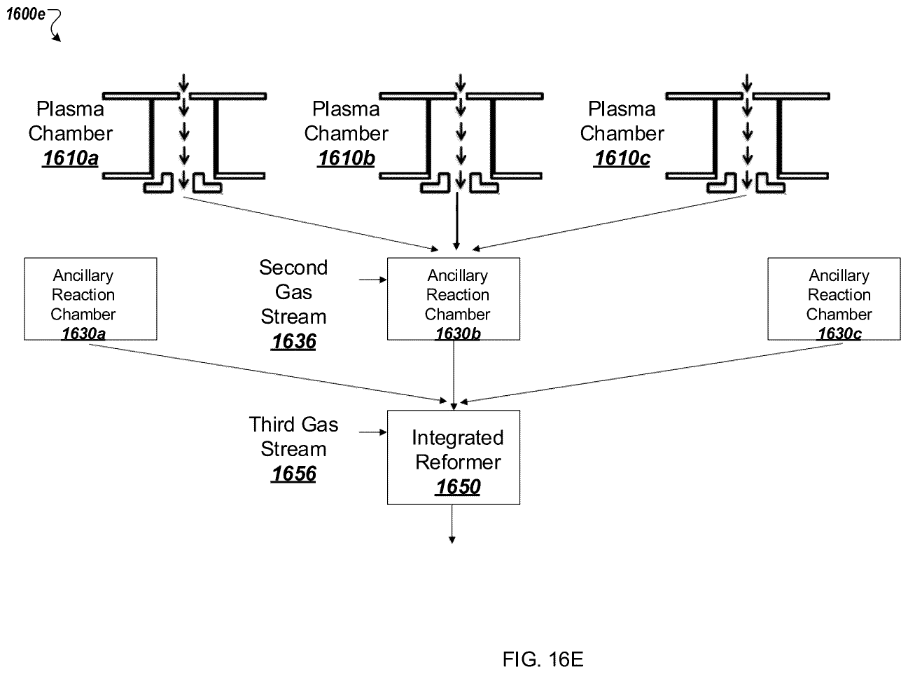

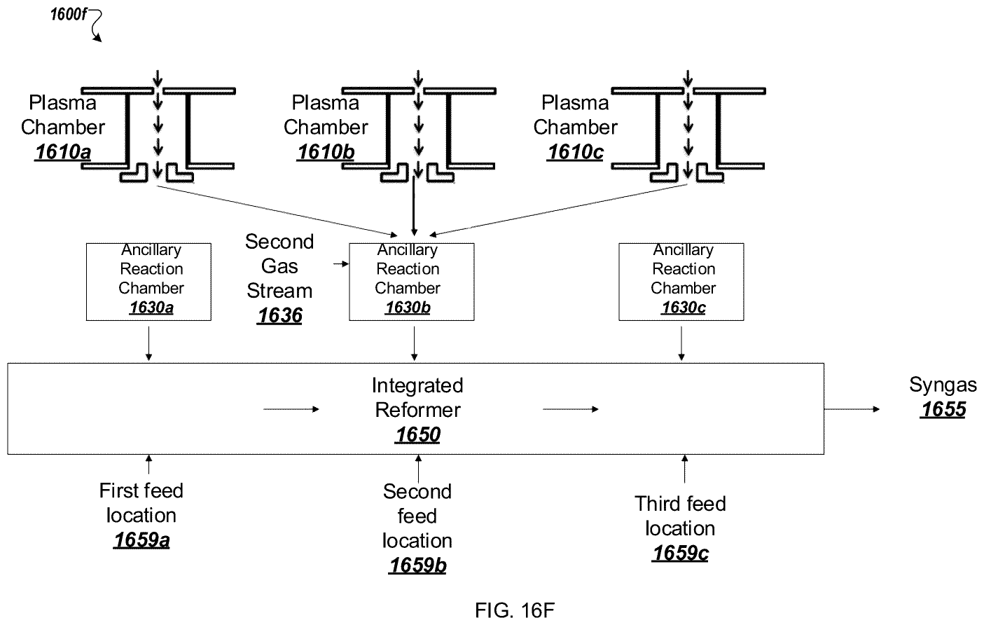

FIG. 16A is a diagram of an example plasma reaction system 1600a that includes a plasma chamber 1610 that may be connected to a first reaction chamber (e.g., an ancillary reaction chamber 1630), and the first reaction chamber may be connected to a second reaction chamber (e.g., an integrated reformer 1650), a third reaction chamber 1652, and a fourth reaction chamber 1654 that may be each connected in parallel with one another. Although the plasma reaction system 1600a is illustrated as having the first reaction chamber in series before the second reaction chamber (e.g., an integrated reformer 1650), the third reaction chamber 1652, and the fourth reaction chamber 1654 in parallel, an outlet flow of the plasma chamber 1610 may first be obtained by the first reaction chamber, the second reaction chamber (e.g., an integrated reformer 1650), the third reaction chamber 1652, and/or the fourth reaction chamber 1654 in parallel in a single serial stage. Additionally or alternatively, one or more reaction chambers may be configured in parallel with each other in a first serial stage with one or more reaction chambers configured in parallel in a second serial stage after the first serial stage such that any number of serial stages with any number of reaction chambers configured in parallel in each serial stage is contemplated. Additionally or alternatively, each reaction chamber that is configured in parallel in a particular serial stage may be simultaneously connected to one or more reaction chambers in a subsequent serial stage and disconnected from one or more other reaction chambers in the same subsequent serial stage.

Various reactor units may be inserted between one or more of the reaction chambers included in a chemical process involving the plasma reaction system 1600a. For example, a non-plasma heat source may be inserted between two serial stages to provide supplemental heat energy to one or more of the reaction chambers. As another example, an integrated reformer, a pressure-swing adsorption unit, an air separation unit, and/or any other reactor units may be implemented to facilitate addition and/or removal of materials from the chemical process. The reaction chambers described herein may include any type of reaction chamber, including an ancillary reaction chamber, or an integrated reformer. Further, the first reaction chamber may not be present and the plasma chamber 1610 may be in fluid communication with the second reaction chamber (e.g., an integrated reformer 1650), the third reaction chamber 1652, and/or the fourth reaction chamber 1654.

Reaction chambers configured in parallel may receive gases flowing at the same or similar flow rates with the same or similar compositions. Consequently, the reaction chambers configured in parallel may operate at the same or similar temperatures and include the same or similar volumes and/or operating pressures. Additionally or alternatively, one or more of the reaction chambers configured in parallel in a particular serial stage may receive gases at a flow rate and/or composition different from the gases received by other reaction chambers in the same particular serial stage. For example, a first pipe directing gases to a first reaction chamber of a particular serial stage may include a greater diameter than a second pipe directing gases to a second reaction chamber of the particular serial stage such that the first reaction chamber receives a greater flowrate of gases than the second reaction chamber.

As illustrated in FIGS. 16A to 16F, a plasma reaction system 1600a, 1600b, 1600c, 1600d, 1600e, 1600f may be configured to include: (i) one or more plasma chambers (e.g., plasma chamber 1610), (ii) one or more ancillary reaction chambers (e.g., ancillary reaction chamber 1630), or (iii) one or more integrated reformers (e.g., integrated reformer 1650). Sensors (not illustrated) may be positioned at any of the features described in conjunction with FIG. 16A to 16F to measure inputs, outputs, energy, utility, and/or parameters related to the plasma reaction system 1600a, 1600b, 1600c, 1600d, 1600e, 1600f and operation thereof.

The one or more plasma chambers may include a single plasma chamber 1610 that may feed into a single ancillary reaction chamber 1630 in series, as illustrated in FIG. 16A. The one or more plasma chambers may include a group of plasma chambers 1610a, 1610b, 1610c that may feed separately into a group of ancillary reaction chambers 1630a, 1630b, 1630c, as illustrated in FIGS. 16B and 16C. The one or more plasma chambers may include a group of plasma chambers 1610a, 1610b, 1610c that may feed into a single ancillary reaction chamber 1630 in series, as illustrated in FIGS. 16D-16F.

The one or more ancillary reaction chambers may include a single ancillary reaction chamber 1630 that may be configured to feed into a group of integrated reformers 1650, as illustrated in FIG. 16A. The one or more ancillary reaction chambers may include a group of ancillary reaction chambers 1630a, 1630b, 1630c that may be configured to feed into a single integrated reformer 1650, as illustrated in FIGS. 16B, 16C, 16E, and 16F. The one or more ancillary reaction chambers may include a group of ancillary reaction chambers 1630a, 1630b, 1630c that may be configured to feed into a single integrated reformer 1650 at distinct feed locations (e.g., first feed location 1659a, second feed location 1659b, third feed location 1659c, or the like), as illustrated in FIGS. 16C and 16F, to generate syngas 1655. The one or more ancillary reaction chambers may include a single ancillary reaction chamber 1630 (e.g., configured to receive a second gas stream 1636) that may be configured to feed into a single integrated reformer 1650 (e.g., configured to receive a third gas stream 1656), as illustrated in FIG. 16D. The one or more ancillary reaction chambers may include a group of ancillary reaction chambers 1630a, 1630b, 1630c that may be configured to feed into a single integrated reformer 1650 at a feed location, as illustrated in FIG. 16E.

The various configurations for the one or more plasma chambers, the one or more ancillary reaction chambers, and the one or more integrated reformers may be selected to maximize the energy conversion efficiency (ECE) (i.e., ECE=product energy/input energy). The one or more ancillary reaction chambers may be configured to receive one or more additional heated first synthesis gas streams from one or more additional plasma chambers (e.g., plasma chambers 1610a, 1610b, 1610c). The one or more integrated reformers may be configured to receive one or more additional heated second synthesis gas streams from one or more additional ancillary reaction chambers (e.g., ancillary reaction chambers 1630a, 1630b, 1630c). The one or more additional heated second synthesis gas streams may be received at one or more additional integrated reformer inlets (e.g., first feed location 1659a, second feed location 1659b, third feed location 1659c, or the like, as illustrated in FIGS. 16C and 16F).

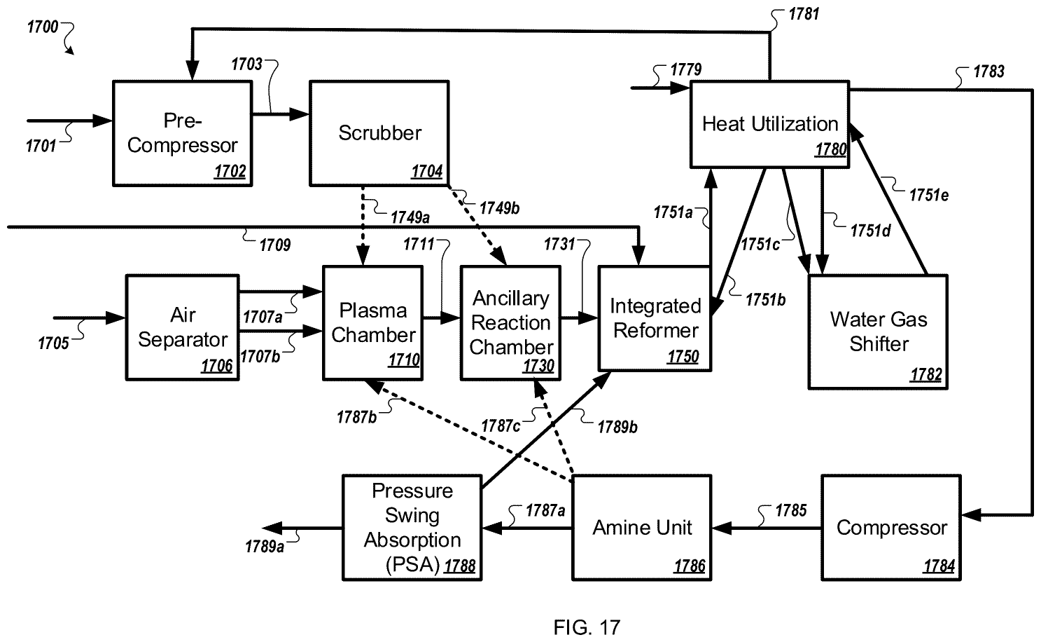

As illustrated in FIG. 17, the system 1700 may include any type of equipment/unit in an environmentally beneficial processes (including, but not limited to) feed gas treatment/scrubber/dryer, carbon capture, O₂ generator (e,g., O₂ PSA, air separation unit), gas tanks, electricity generator, heat exchanger, C₂H₂ removal, water-gas shift, CO₂ capture, syngas separator, product gas purification (syngas, H₂, and/or CO), syngas processes/transformations (e.g., CO₂ hydrogenation, methanol, ethanol, ammonia, fertilizer, plastics, additives, any decarbonized material, sustainable aviation fuel (SAF), fuels, acids, chemicals), H₂ compression and liquefaction, electrolyzer (high- and low-temperature electrolyzers that may utilize heat and water/steam), steam methane reforming (e.g., SMR), water treatment, drain, particulate collection, boiler, flare, oxidizer, steam export pipeline, CO₂ tanks, etc. Some or all of these equipment/units may be monitored and all inputs, outputs, energy, utility, and parameters may be measured and used for tokenization.

FIG. 17 is a diagram of a plasma reaction system 1700 for carbon dioxide utilization using a PCCU. Sensors (not illustrated) may be positioned at any of the features described in conjunction with FIG. 17 to measure inputs, outputs, energy, utility, and/or parameters related to the plasma reaction system 1700 and operation thereof. The carbon dioxide utilization system may include a pre-compressor unit 1702 into which biogas may be inputted to increase the pressure of the input biogas 1701 that may contain methane and carbon dioxide. Increasing the pressure of the input biogas 1701 may be facilitated by directing power 1781 from a heat utilization unit 1780 to the pre-compressor unit 1702. For example, the input biogas 1701 may be obtained by the pre-compressor unit 1702 at a pressure of 0.5 atm, 1 atm, 1.5 atm, 2 atm, or some other pressure, and the biogas 1703 exiting the pre-compressor unit 1702 may be at a pressure of 2 atm, 3 atm, 5 atm, 10 atm, 20 atm, 100 atm, or some other pressure. The pressurized biogas 1703 may be sent to a scrubber 1704 that may remove impurities, pollutants, or otherwise harmful components of the biogas. For example, the scrubber 1704 may include a dry scrubbing process in which harmful substances (e.g., sulfur oxides, particulate matter, acidic gases, etc.) may be adsorbed to dry reagents included in the scrubber 1704. As another example, the scrubber 1704 may include a wet scrubbing process in which the biogas 1703 is sprayed with a wet substance (e.g., water) to separate one or more components from the biogas 1703.

An air separator 1706 may obtain an input air stream 1705 and separate the input air stream 1705 into its constituent components, which may primarily include nitrogen gas and oxygen gas. The air separator 1706 may facilitate separation of the components included in the input air stream 1705 via one or more of fractional distillation, pressure swing adsorption, vacuum pressure swing adsorption, membrane separation, or by any other separation methods. The separated air components 1707a, 1707b may be sent to a plasma chamber 1710 of a PCCU that may include one or more of a plasma chamber 1710, an ancillary reaction chamber 1730, or an integrated reformer 1750.

The scrubbed biogas 1749a may be sent to the plasma chamber 1710 and the ancillary reaction chamber 1730 of the PCCU. The plasma reactor may include a plasma chamber 1710 made of a quartz or ceramic material in which one or more waveguides are configured to facilitate chemical reactions that occur in the plasma chamber 1710. Fuel (e.g., hydrocarbon such as natural gas or methane) and other input compounds heated by electricity or microwaves may react to provide more heat to input compounds of the plasma chamber 1710. The plasma chamber 1710 may be configured to obtain an inlet stream of scrubbed biogas 1749a, the separated air components 1707a, 1707b, and carbon dioxide 1787b from an amine unit 1786 to affect chemical reactions between the inlet streams of gases. For example, incoming scrubbed biogas 1749a may react with the incoming carbon dioxide 1787b and oxygen 1707b at high temperatures provided by the electrically heated plasma to form syngas 1711 and excess heat that are then sent to the ancillary reaction chamber 1730.

The ancillary reaction chamber 1730 may be configured to obtain syngas 1711 from the plasma chamber 1710, scrubbed biogas 1749b, and carbon dioxide 1787c from an amine unit 1786 to affect chemical reactions between the inlet stream of gases. For example, in the ancillary reaction chamber 1730, incoming scrubbed biogas 1749b may react with the incoming carbon dioxide 1787c at high temperatures provided by the heat generated (e.g., from an exothermic reaction) by the plasma chamber 1710 to form syngas 1731 and excess heat that are then sent to the integrated reformer 1750. In this and other examples, the oxygen gas obtained from the air separator 1706 may facilitate reaction between the scrubbed biogas and the carbon dioxide.

The integrated reformer 1750 may include a steam methane reforming reactor (SMR) or any other reactor vessel that may be configured to convert hydrocarbon included in the natural gas 1709 into hydrogen and carbon monoxide using electrically generated or microwave generated heat (e.g., the heat provided by the plasma chamber 1710) and chemical reaction heat (e.g., heat provided by the plasma chamber and ancillary reaction chamber) rather than combusted natural gas or other fuels. Additionally or alternatively, the integrated reformer 1750 may be configured to generate additional syngas using natural gas 1709. The integrated reformer 1750 may be configured to obtain a recycled stream of steam 1751b from the heat utilization unit 1780 in which the recycle streams provide additional reactants to facilitate a greater conversion rate of hydrocarbon and biogas into one or more product gases.

The syngas 1751a produced by the integrated reformer 1750 may be sent to the heat utilization unit 1780, which may yield a cooled product gas 1751c and steams of steam 1751b and 1751d. The heat utilization unit 1780 may include a steam-generating or a power-generating unit that may be configured to receive an input stream of water 1779 and the syngas 1751a generated by the integrated reformer 1750. The heat utilization unit 1780 may vaporize the input water 1779 using the excess heat generated by the PCCU and input to the heat utilization unit 1780 by the incoming syngas 1751a stream from the integrated reformer 1750, and the generated steam 1751d may be sent to a water-gas shifter (WGS) 1782. Some of the syngas 1751a sent to the heat utilization unit 1780 from the integrated reformer 1750 may be routed to the WGS 1782 (1751c). The WGS 1782 may facilitate formation of hydrogen gas via a water-gas shift reaction in which carbon monoxide and water reversibly react to form carbon dioxide and hydrogen that are sent back to the heat utilization unit 1780 (e.g., using line 1751e). Additionally or alternatively, any excess steam 1751b from the heat utilization unit 1780 may be sent to the integrated reformer 1750 to recycle any excess heat to facilitate the formation of the syngas 1751a in the integrated reformer 1750.

The hydrogen gas and any unreacted or partially reacted materials may be sent from the heat utilization unit 1780 to a compressor 1784 that pressurizes the input materials 1783 and sends the output gases 1785 to an amine unit 1786. Increasing the pressure of the input materials 1783 may be facilitated by excess power obtained from the heat utilization unit 1780. For example, the input materials 1783 may be obtained by the compressor at a pressure of 0.5 atm, 1 atm, 1.5 atm, 2 atm, or some other pressure, and the hydrogen gas and/or any other gases exiting the compressor 1784 may be at a pressure of 2 atm, 3 atm, 5 atm, 10 atm, 20 atm, 100 atm, or some other pressure.

The amine unit 1786 may include various aqueous solutions of amines that may react with the gases 1785 exiting the compressor 1784 to remove any remaining impurities (e.g., hydrogen sulfide, sulfur oxides, or any other harmful substances). Additionally or alternatively, the amines included in the amine unit 1786 may facilitate removal of acidic gases, such as the carbon dioxide. The carbon dioxide 1787b, 1787c removed by the amine unit 1786 may be recycled and sent back to the plasma chamber 1710 and/or the ancillary reaction chamber 1730 to facilitate further syngas reactions.

The remaining hydrogen gas and any residual gases from the amine unit 1786 may be sent to a pressure swing adsorption unit 1788 (PSA) to further separate the obtained gases 1787a to generate a high purity H₂ 1789a. For example, the pressure swing adsorption unit 1788 may include a membrane of adsorbent materials that may separate the hydrogen gas from any other gases that entered the pressure swing adsorption unit 1788 by catching compounds passing through the membrane aside from the hydrogen gas. The gases caught by the membrane may be desorbed from the adsorbent materials by reducing the pressure in the pressure swing adsorption unit 1788, and the desorbed gases 1789b may be recycled into the PCCU (e.g., to the integrated reformer 1750) for further reacting.

Modifications, additions, or omissions may be made to the system of carbon dioxide utilization without departing from the scope of the present disclosure. For example, the designations of different elements in the manner described is meant to help explain concepts described herein and is not limiting. For instance, in some embodiments, the pre-compressor unit 1702, scrubber 1704, air separator 1706, PCCU including the plasma chamber 1710, the ancillary reaction chamber 1730, and the integrated reformer 1750, heat utilization unit 1780, WGS 1782, compressor 1784, amine unit 1786, and pressure swing adsorption unit 1788 are delineated in the specific manner described to help with explaining concepts described herein but such delineation is not meant to be limiting. Further, the system of carbon dioxide utilization may include any number of other elements or may be implemented within other systems or contexts than those described.

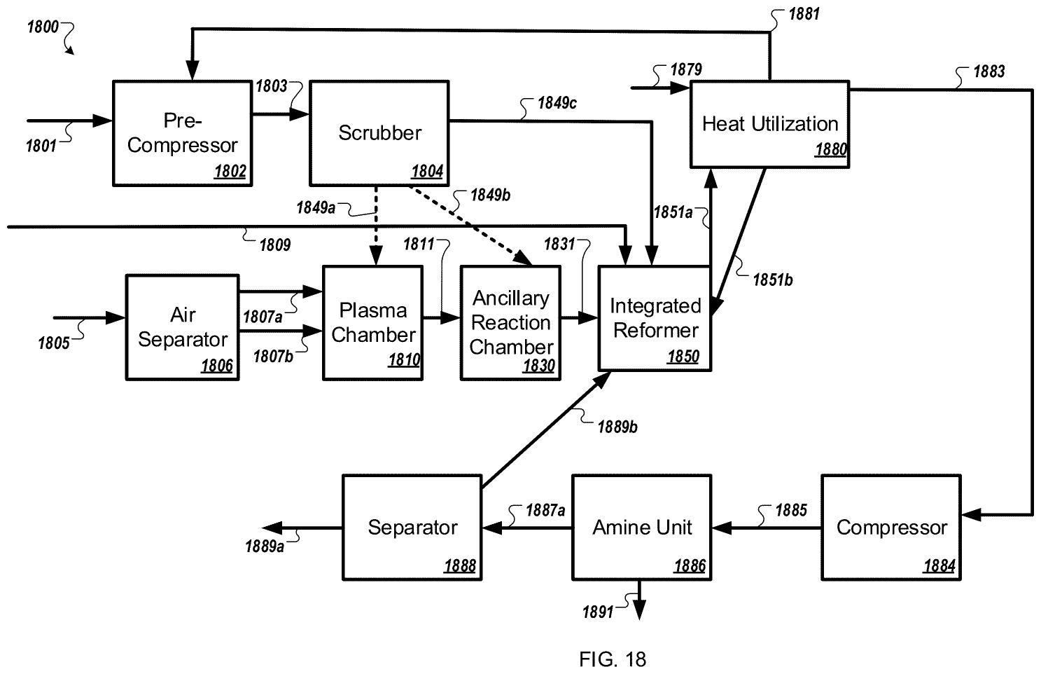

FIG. 18 is a diagram of a plasma reaction system 1800 of synthesizing hydrogen gas and carbon monoxide using a PCCU. Sensors (not illustrated) may be positioned at any of the features described in conjunction with FIG. 18 to measure inputs, outputs, energy, utility, and/or parameters related to the plasma reaction system 1800 and operation thereof. The carbon dioxide utilization system may include a pre-compressor unit 1802 into which biogas may be inputted to increase the pressure of the input biogas 1801 that may contain methane and carbon dioxide. Increasing the pressure of the input biogas 1801 may be facilitated by directing power 1881 from a heat utilization unit 1880 to the pre-compressor unit 1802. For example, the input biogas 1801 may be obtained by the pre-compressor unit 1802 at a pressure of 0.5 atm, 1 atm, 1.5 atm, 2 atm, or some other pressure, and the biogas 1803 exiting the pre-compressor unit 1802 may be at a pressure of 2 atm, 3 atm, 5 atm, 10 atm, 20 atm, 100 atm, or some other pressure. The pressurized biogas 1803 may be sent to a scrubber 1804 that may remove impurities, pollutants, or otherwise harmful components of the biogas. For example, the scrubber 1804 may include a dry scrubbing process in which harmful substances (e.g., sulfur oxides, particulate matter, acidic gases, etc.) are adsorbed to dry reagents included in the scrubber 1804. As another example, the scrubber 1804 may include a wet scrubbing process in which the biogas is sprayed with a wet substance (e.g., water) to separate one or more components from the biogas 1803. The scrubbed biogas 1849a may be sent to a plasma chamber 1810, an ancillary reaction chamber 1830, and/or an integrated reformer 1850 of a PCCU.

An air separator 1806 may obtain an input air stream 1805 and separate the input air stream 1805 into its constituent components, which may primarily include nitrogen gas and oxygen gas. The air separator 1806 may facilitate separation of the components included in the input air stream 1805 via fractional distillation, pressure swing adsorption, vacuum pressure swing adsorption, membrane separation, or by any other separation methods. The separated air components 1807a, 1807b may be sent to the PCCU that may include the plasma chamber 1810, the ancillary reaction chamber 1830, and/or the integrated reformer 1850.

The plasma chamber 1810 may be made of a quartz or ceramic material in which one or more waveguides may be configured to facilitate chemical reactions that may occur in the plasma chamber 1810. Fuel (e.g., hydrocarbon) and other input compounds heated by electricity or microwaves may react to provide more heat to input compounds of the plasma reactor. The plasma chamber 1810 may be configured to obtain an inlet stream of the separated air components 1807a, 1807b and scrubbed biogas 1849a from the scrubber. The oxygen gas obtained from the air separator 1806 and the energy provided in the plasma reactor may facilitate conversion of the scrubbed biogas 1849a into syngas 1811.

The ancillary reaction chamber 1830 may be configured to obtain syngas 1811 from the plasma chamber 1810 and scrubbed biogas 1849b to affect chemical reactions between the inlet stream of gases. For example, in the ancillary reaction chamber 1830, incoming biogas 1849b may react at high temperatures provided by the heat generated (e.g., from an exothermic reaction) by the plasma chamber 1810 to form syngas 1831 and excess heat that may be sent to the integrated reformer 1850.

The integrated reformer 1850 may include a steam methane reforming reactor (SMR) or any other reactor vessel that is configured to obtain the scrubbed biogas 1849c from the scrubber 1804 and/or natural gas 1809, and convert it into syngas 1851a using electrically generated or microwave-generated heat and chemical reaction heat (e.g., the heat provided by one or more of the plasma chamber 1810 or the ancillary reaction chamber 1830). Additionally or alternatively, the integrated reformer 1850 may be configured to obtain a recycle stream of waste gas from a separator unit 1888 and/or a recycled stream of steam 1851b from the heat utilization unit 1880 in which the recycle streams from the separator unit 1888 and the heat utilization unit 1880 provide additional reactants to facilitate a greater conversion rate of biogas into syngas 1851a. The syngas 1851a yielded by chemical reactions occurring in the PCCU may be generated more efficiently than syngas yielded by other existing chemical processes. Additionally or alternatively, additional heat may not be used to facilitate the syngas reactions occurring in the integrated reformer 1850 because the heat generated by the reactions occurring in the plasma chamber 1810 and/or the ancillary reaction chamber 1830 may be inputted into the integrated reformer 1850.

The syngas 1851a produced by the integrated reformer 1850 may be sent to the heat utilization unit 1880, which may yield one or more product gases 1883 derived from the syngas 1851a. The heat utilization unit 1880 may include a steam-generating or a power-generating unit that may be configured to receive an input stream of water 1879 and the syngas 1851a generated by the integrated reformer 1850. The heat utilization unit 1880 may vaporize the input water 1879 using the excess heat generated by the PCCU and input to the heat utilization unit 1880 by the incoming syngas 1851a stream from the integrated reformer 1850, and the generated steam 1851b may facilitate production of the syngas product gases 1851a in the integrated reformer 1850.

Any excess steam 1851b from the heat utilization unit 1880 may be sent to the integrated reformer 1850 to recycle any excess heat to facilitate the formation of the syngas 1851a in the integrated reformer 1850. The product gases yielded from the syngas 1851a may include hydrogen gas and carbon monoxide in a ratio ranging from 0.5:1 to 2.9:1. The ratio of product gases from the heat utilization unit 1880 may be dependent on a volume and composition of input biogas 1801 into the pre-compressor unit 1802, a volume of input air stream 1805 into the air separator 1806, an amount of energy supplied to the plasma chamber 1810 of the PCCU, an amount of steam 1851b sent to the integrated reformer 1850 from the heat utilization unit 1880, or some combination thereof. For example, a ratio of hydrogen gas to carbon monoxide may range from approximately 0.5:1 to approximately 1.5:1 when there is no recycle stream of steam 1851b from the heat utilization unit 1880 to the integrated reformer 1850, while the ratio of hydrogen gas to carbon monoxide may increase to approximately 1.3:1 to approximately 2.9:1 depending on the amount of steam 1851b recycled to the integrated reformer 1850.

The product gases 1883 and any unreacted or partially reacted materials may be sent from the heat utilization unit 1880 to a compressor 1884 that pressurizes the product gases 1883 (and any other input materials) and outputs pressurized product gases 1885 to an amine unit 1886. Increasing the pressure of the product gases 1883 may be facilitated by excess heat obtained from the heat utilization unit 1880. For example, the product gases 1883 may be obtained by the compressor 1884 at a pressure of 0.5 atm, 1 atm, 1.5 atm, 2 atm, or some other pressure, and the hydrogen gas and/or any other gases exiting the compressor 1884 may be at a pressure of 2 atm, 3 atm, 5 atm, 10 atm, 20 atm, 100 atm, or some other pressure.

The amine unit 1886 may include various aqueous solutions of amines that may react with the pressurized product gases 1885 exiting the compressor 1884 to remove any remaining impurities (e.g., hydrogen sulfide, sulfur oxides, or any other harmful substances). Additionally or alternatively, the amines included in the amine unit 1886 may facilitate removal of acidic gases, such as the carbon dioxide. The carbon dioxide 1891 removed by the amine unit 1886 may be sent to another reactor unit or processing system, such as the system of carbon dioxide utilization.

The pressurized product gases 1885 treated at the amine unit 1886 may be sent to a separator unit 1888 that splits the various components of the product gases 1887a. As such, any partially reacted or unreacted gases that entered the separator unit 1888 alongside the product gases may be redirected back to the integrated reformer 1850 in a recycle stream 1889b to drive the chemical reactions relating to formation of the syngas 1851a to a further degree of completion. Additionally or alternatively, the separator unit 1888 may include a fractional distillation system, a vacuum pressure swing adsorption system, or any other separation processes that may be designed to separate the product gases 1889a (e.g., H₂ and CO) from unreacted or partially reacted component gases. The separated product gases 1889a may also be divided from one another such that different product gases may be directed to different locations (e.g., different storage containers).

Modifications, additions, or omissions may be made to the system of synthesizing hydrogen gas and carbon monoxide without departing from the scope of the present disclosure. For example, the designations of different elements in the manner described is meant to help explain concepts described herein and is not limiting. For instance, in some embodiments, the pre-compressor unit 1802, scrubber 1804, air separator 1806, PCCU including the plasma chamber 1810, the ancillary reaction chamber 1830, and/or the integrated reformer 1850, heat utilization unit 1880, compressor 1884, amine unit 1886, and separator unit 1888 may be delineated in the specific manner described to help with explaining concepts described herein but such delineation is not meant to be limiting. Further, the system of synthesizing hydrogen gas and carbon monoxide may include any number of other elements or may be implemented within other systems or contexts than those described.

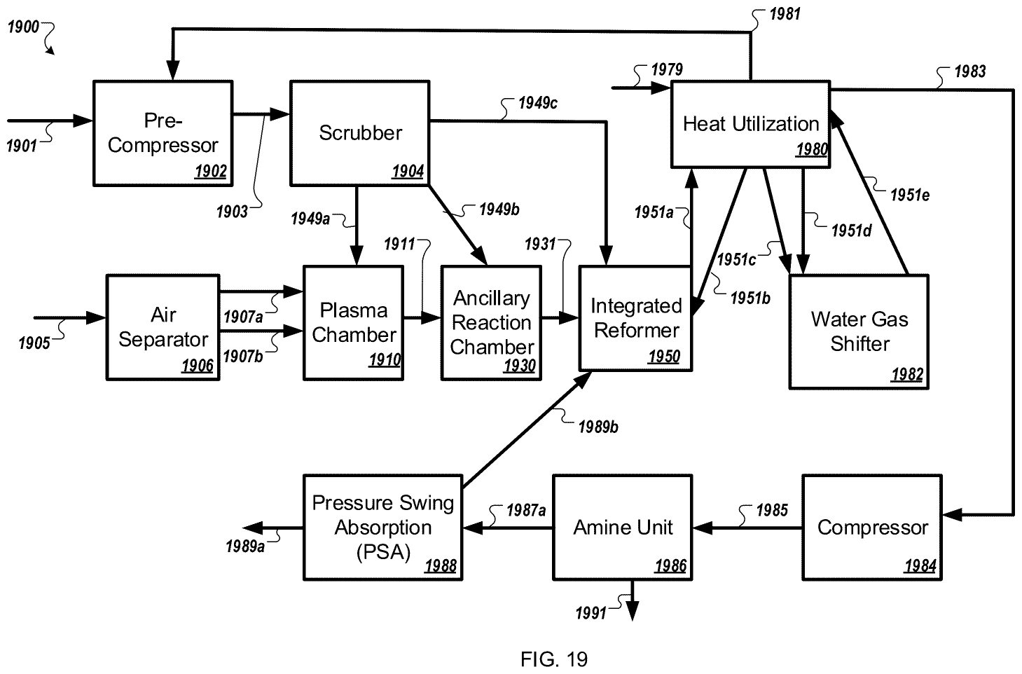

FIG. 19 is a diagram of a plasma reaction system 1900 of converting biogas into hydrogen gas using a PCCU. Sensors (not illustrated) may be positioned at any of the features described in conjunction with FIG. 19 to measure inputs, outputs, energy, utility, and/or parameters related to the plasma reaction system 1900 and operation thereof. The carbon dioxide utilization system may include a pre-compressor unit 1902 into which input biogas 1901 may be inputted. Increasing the pressure of the input biogas 1901 may be facilitated by directing power from a heat utilization unit 1980 to the pre-compressor unit 1902. For example, the input biogas 1901 may be obtained by the pre-compressor unit 1902 at a pressure of 0.5 atm, 1 atm, 1.5 atm, 2 atm, or some other pressure, and the biogas 1903 exiting the pre-compressor unit 1902 may be at a pressure of 2 atm, 3 atm, 5 atm, 19 atm, 20 atm, 100 atm, or some other pressure. The pressurized biogas 1903 may be sent to a scrubber 1904 that may remove impurities, pollutants, or otherwise harmful components of the biogas. For example, the scrubber 1904 may include a dry scrubbing process in which harmful substances (e.g., sulfur oxides, particulate matter, acidic gases, etc.) may be adsorbed to dry reagents included in the scrubber 1904. As another example, the scrubber 1904 may include a wet scrubbing process in which the biogas is sprayed with a wet substance (e.g., water) to separate one or more components from the biogas 1903.

An air separator 1906 may obtain an input air stream 1905 and separate the input air stream 1905 into its constituent components 1907a, 1907b, which may primarily include nitrogen gas 1907a and oxygen gas 1907b. The air separator 1906 may facilitate separation of the components included in the input air stream 1905 via fractional distillation, pressure swing adsorption, vacuum pressure swing adsorption, membrane separation, or by any other separation methods. The separated air components 1907a, 1907b may be sent to a PCCU that may include a plasma chamber 1910, an ancillary reaction chamber 1930, and an integrated reformer 1950.

The plasma chamber 1910 may be made of a quartz or ceramic material in which one or more waveguides are configured to facilitate chemical reactions that occur in the plasma chamber 1910. Fuel (e.g., hydrocarbon) and other input compounds heated by electricity or microwaves may react to provide more heat to input compounds of the plasma chamber 1910. The plasma chamber 1910 may be configured to obtain an inlet stream of the separated air components 1907a, 1907b and scrubbed biogas 1949a from the scrubber 1904. The oxygen gas obtained from the air separator 1906 and the energy provided in the plasma chamber 1910 may facilitate conversion of the scrubbed biogas 1949a into syngas 1911.

The ancillary reaction chamber 1930 may be configured to obtain syngas 1911 from the plasma chamber 1910 to affect chemical reactions between the inlet stream of gases. For example, in the ancillary reaction chamber 1930, incoming scrubbed biogas 1949b may react at high temperatures provided by the heat generated (e.g., from an exothermic reaction) by the plasma chamber 1910 to form syngas 1931 and excess heat that may be sent to the integrated reformer 1950.

The integrated reformer 1950 may be a discrete reaction unit that may be connected to the ancillary reaction chamber 1930. The integrated reformer 1950 may include a steam methane reforming reactor (SMR) or any other reactor vessel that is configured to obtain the scrubbed biogas 1949c from the scrubber 1904 and convert it into syngas 1951a using electrically generated or microwave-generated heat and chemical reaction heat (e.g., the heat provided by the plasma chamber 1910 and/or the ancillary reaction chamber 1930). Additionally or alternatively, the integrated reformer 1950 may be configured to obtain a recycle stream 1989b of syngas from a PSA unit 1988 and/or a recycled stream of steam 1951b from the heat utilization unit 1980 in which the recycle streams from the PSA unit 1988 and the heat utilization unit 1980 provide additional reactants to facilitate a greater conversion rate of biogas into syngas 1951a.

The syngas 1951a produced by the integrated reformer 1950 may be sent to the heat utilization unit 1980, which yields one or more cooled product gases (e.g., syngas 1951c) derived from the syngas 1951a and 1983 derived from the syngas 1951e. The heat utilization unit 1980 may include a steam-generating or a power-generating unit that may be configured to receive an input stream of water 1979, the syngas 1951a generated by the integrated reformer 1950, and the syngas 1951e from a water gas shifter (WGS). The heat utilization unit 1980 may vaporize the input water 1979 using the excess heat generated by the PCCU and input to the heat utilization unit 1980 by the incoming syngas 1951a stream from the integrated reformer 1950, and the generated steam 1951b may facilitate conversion of the biogas 1949c into the product gases (e.g., syngas 1951a). Additionally or alternatively, any excess steam 1951b from the heat utilization unit 1980 may be sent to the integrated reformer 1950 to recycle the heat to facilitate further formation of the syngas 1951a in the integrated reformer 1950.

The heat utilization unit 1980 may vaporize the water 1979 using the excess heat generated by the PCCU and input to the heat utilization unit 1980 by the incoming syngas 1951a stream from the integrated reformer 1950, and the generated steam 1951d and syngas 1951c may be sent to a water-gas shifter (WGS) 1982. Additionally or alternatively, excess steam 1951b from the heat utilization unit 1980 may be sent to the integrated reformer 1950 to recycle the excess heat to facilitate the formation of the syngas 1951a in the integrated reformer 1950.

Some of the syngas 1951a sent to the heat utilization unit 1980 from the integrated reformer 1950 may be routed to the WGS 1982 (1951c). The WGS 1982 may facilitate formation of hydrogen gas via a water-gas shift reaction in which carbon monoxide and water reversibly react to form carbon dioxide and hydrogen gas that are sent back to the heat utilization unit 1980 (1951e).

In these and other embodiments, the product gases 1983 (e.g., the hydrogen gas generated in the WGS 1982) and any unreacted or partially reacted materials may be sent from the heat utilization unit 1980 to a compressor 1984 that pressurizes the product gases (and any other input materials) and sends the pressurized product gases 1985 to an amine unit 1986. Increasing the pressure of the product gases 1983 may be facilitated by excess power 1981 obtained from the heat utilization unit 1980. For example, the product gases 1983 may be obtained by the compressor at a pressure of 0.5 atm, 1 atm, 1.5 atm, 2 atm, or some other pressure, and the pressurized product gases 1985 (e.g., hydrogen gas and/or any other gases) exiting the compressor 1984 may be at a pressure of 2 atm, 3 atm, 5 atm, 10 atm, 20 atm, 100 atm, or some other pressure.

The amine unit 1986 may include various aqueous solutions of amines that react with the pressurized product gases 1985 exiting the compressor 1984 to remove any remaining impurities (e.g., hydrogen sulfide, sulfur oxides, or any other harmful substances). Additionally or alternatively, the amines included in the amine unit 1986 may facilitate removal of acidic gases, such as the carbon dioxide. The carbon dioxide 1991 removed by the amine unit 1986 may be sent to another reactor unit or processing system, such as the system of carbon dioxide utilization.

The product gases 1987a treated at the amine unit 1986 may be sent to the PSA unit 1988 that splits the various components of the product gases 1987a. For example, the PSA unit 1988 may separate the components included in the product gases in which the PSA unit 1988 includes a membrane of adsorbent materials that separates gas components that entered the PSA unit 1988 by filtering compounds passing through the membrane. The gases caught by the membrane may be desorbed from the adsorbent materials by reducing the pressure in the PSA unit 1988, and the desorbed gases may be recycled into the PCCU (e.g., to the integrated reformer 1950) for further reacting. As such, any partially reacted or unreacted gases that entered the separator unit alongside the product gases may be redirected back to the integrated reformer 1950 in a recycle stream 1989b to drive the chemical reactions relating to formation of the syngas 1951a to a further degree of completion. Additionally or alternatively, the separated product gases may be divided from one another such that different product gases 1989a may be directed to different locations (e.g., different storage containers).

Modifications, additions, or omissions may be made to the system of converting biogas into hydrogen gas without departing from the scope of the present disclosure. For example, the designations of different elements in the manner described is meant to help explain concepts described herein and is not limiting. For instance, in some embodiments, the pre-compressor unit 1902, scrubber 1904, air separator 1906, PCCU including the plasma chamber 1910, the ancillary reaction chamber 1930, and/or the integrated reformer 1950, heat utilization unit 1980, WGS 1982, compressor 1984, amine unit 1986, and PSA Unit 1988 are delineated in the specific manner described to help with explaining concepts described herein but such delineation is not meant to be limiting. Further, the system of converting biogas into hydrogen gas may include any number of other elements or may be implemented within other systems or contexts than those described.

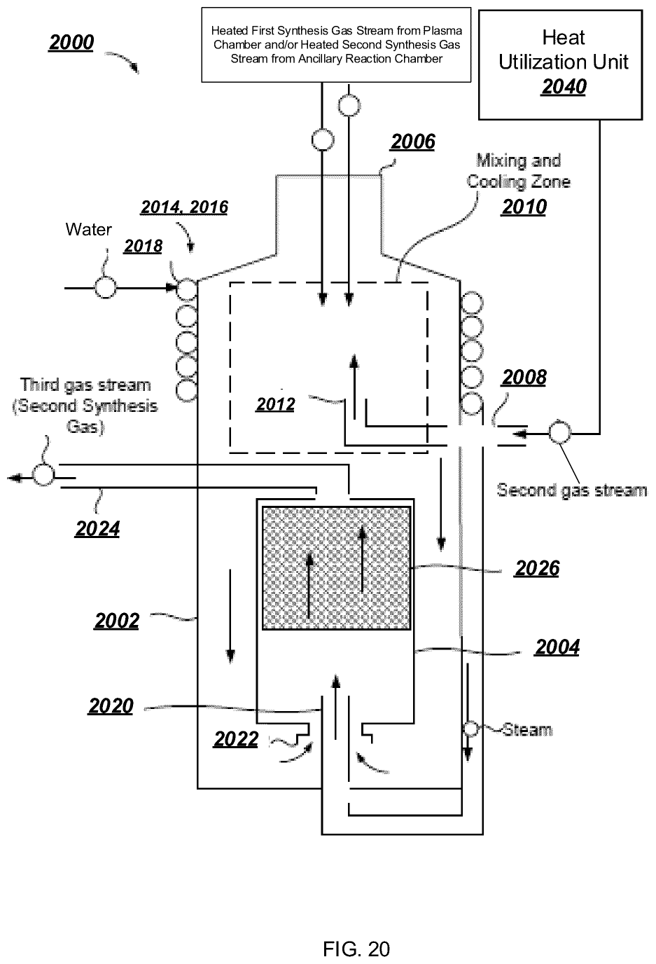

FIG. 20 illustrates an example of an integrated reformer 2000. The integrated reformer 2000 may obtain one or more gaseous compounds from a plasma chamber. Sensors (not illustrated) may be positioned at any of the features described in conjunction with FIG. 20 to measure inputs, outputs, energy, utility, and/or parameters related to the integrated reformer and operation thereof. The gaseous compounds obtained from the plasma chamber may be heated to a high temperature due to exothermic reactions in the plasma chamber. As such, excess heat from the plasma chamber may be transferred to the integrated reformer 2000 by the gaseous compounds outputted by the plasma chamber. In some embodiments, the integrated reformer 2000 may include an outer chamber and an inner reaction chamber in which the gaseous compounds flow from the outer chamber into the inner reaction chamber. Additionally or alternatively, water and/or steam may be fed into the integrated reformer 2000 such that the water and/or the steam flow from the outer chamber into the inner reaction chamber or are fed directly into the inner reaction chamber. The integrated reformer 2000 may output syngas using outlet 2024 or other gaseous products, which may be sent to one or more processing units to further treat the gaseous products.

FIG. 20 illustrates a cross-sectional view of an example integrated reformer 2000. The integrated reformer 2000 may include an outer chamber 2002 and the reaction chamber 2004. The outer chamber 2002 may include a first inlet 2006 configured to obtain a first gas stream (e.g., gas stream including a heated second synthesis gas stream from an ancillary reaction chamber) and a second inlet 2008 configured to obtain a second gas stream (e.g., gas stream including hydrocarbon fuel such as biogas, CH₄, natural gas, or any combinations thereof). The second gas stream may be from a heat utilization unit 2040. The second gas stream may be heated or cooled by the heat utilization unit 2040 to a pre-determined temperature before being directed to the outer chamber 2002.

The integrated reformer 2000 may provide a mixing and cooling zone 2010 in the outer chamber 2002 where the first gas stream and the second gas stream may be mixed and cooled. To mix the first gas stream and the second gas stream together in the mixing and the cooling zone 2010, a first end 2012 of the second inlet 2008 may be extended into the mixing and cooling zone 2010 via a wall of the outer chamber 2002. The first end 2012 of the second inlet 2008 in the outer chamber 2002 may be bent so that the first end 2012 of the second inlet 2008 may be directed to the first inlet 2006. The first gas stream and the second gas stream may collide directly at the mixing and the cooling zone 2010 for better mixing.

The first gas stream may be from a plasma chamber and/or an ancillary reaction chamber (including the heated second synthesis gas stream 1435 from the ancillary reaction chamber, as described in relation to FIG. 14). The first output stream (e.g., first gas stream in FIG. 14) from the plasma chamber may have a high temperature (e.g., temperature range between 1500° C. and 2500° C.) due to operations in the plasma chamber.

By mixing the first gas stream (with the high temperature) with the second gas stream (biogas in this example) with a temperature relatively lower than the first gas stream, the temperature of the mixture of the first gas stream and the second gas stream may be lower than the temperature of the first gas stream. To efficiently cool down the mixture of the first gas stream and the second gas stream to a predetermined temperature or a predetermined temperature range so that the temperature of the mixture of the first gas stream and the second gas stream is within a range (e.g., between 700° C. and 1000° C.) suitable for steam reforming (in the reaction chamber 2004 along with steam), a cooling unit 2014 may be coupled to the outer chamber 2002.

The integrated reformer 2000 may include the cooling unit 2014 disposed adjacent to mixing and cooling zone 2010. The cooling unit 2014 may surround the mixing and cooling zone 2010. The cooling unit 2014 may include a tube 2016 (or a pipe) disposed or wrapped around the outer chamber 2002 adjacent to the mixing and cooling zone 2010. The mixing and cooling zone 2010 may be positioned between the first inlet 2006 and the reaction chamber 2004.

The cooling unit 2014 may use water as coolant to cool down the mixture of the first gas stream and/or the second gas stream. For example, water may be supplied to a first end 2018 of the tube 2016 disposed around the outer chamber 2002. As the water flows within the tube 2016, the water may absorb the thermal energy (heat) from the mixture of the first gas stream and/or the second gas stream, and becomes steam (e.g., water in gas state).

The cooling unit 2014 may be configured to provide the steam to the reaction chamber 2004. A second end 2020 of the tube 2016 may be disposed in the reaction chamber 2004 adjacent to a mixture gas inlet 2022 of the reaction chamber 2004. The second end 2020 of the tube 2016 may be connected to (or in fluid communication with) the reaction chamber 2004 via the mixture gas inlet 2022 of the reaction chamber 2004.

The second end 2020 of the tube 2016 may be directed to a side opposite to the side of the mixture gas inlet 2022. The mixture of the first gas stream and the second gas stream may be provided to the reaction chamber 2004 via the mixture gas inlet 2022. As a result, the mixture of the first gas stream and the second gas stream may be mixed with the steam in the reaction chamber 2004. As a result, the reaction chamber 2004 may generate a third gas stream based on the first gas stream, the second gas streams, and the steam using the steam reforming. The reaction chamber 2004 may include an outlet 2024 to output the third gas stream (including the syngas) generated by the integrated reformer 2000. The reaction chamber 2004 may include a catalyst 2026 to promote more reactions for synthesis gas production (e.g., catalytic process). The catalyst may include a porous material or structure (e.g., mesh, group of tubes or pipes, membrane). The catalyst 2026 may be disposed between the outlet 2024 and the mixture gas inlet 2022 so the mixtures of the first gas stream, the second gas steam, and the steam from the cooling unit 2014 may efficiently pass through the catalyst 2026 for catalytic process.

Modifications, additions, or omissions may be made to the integrated reformer 2000 without departing from the scope of the present disclosure. For example, the coolant in the cooling unit 2014 may be used for cooling the mixture of the first gas stream and/or the second gas stream and steam from the heat utilization unit used for the steam reforming process. The coolant, after cooling the mixture of the first gas stream and/or the second stream, may be re-used by the cooling unit 2014 after condensing.

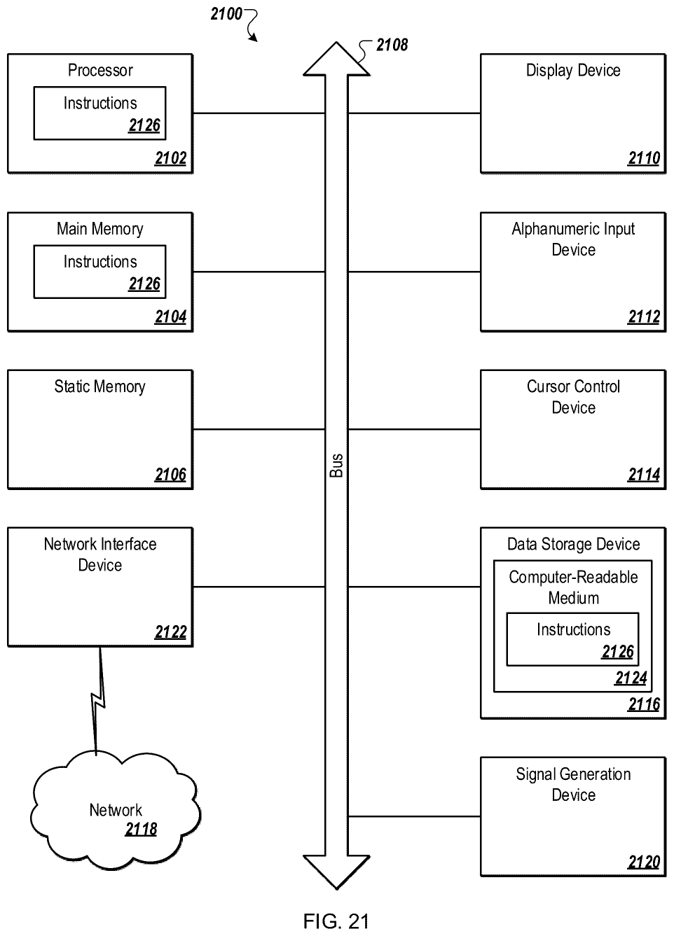

FIG. 21 illustrates a diagrammatic representation of a machine in the example form of a computing device 2100 within which a set of instructions, for causing the machine to perform any one or more of the methods discussed herein, may be executed. The computing device 2100 may include a rackmount server, a router computer, a server computer, a mainframe computer, a laptop computer, a tablet computer, a desktop computer, or any computing device with at least one processor, etc., within which a set of instructions, for causing the machine to perform any one or more of the methods discussed herein, may be executed. In alternative examples, the machine may be connected (e.g., networked) to other machines in a local area network (LAN), an intranet, an extranet, or the Internet. The machine may operate in the capacity of a server machine in client-server network environment. Further, while only a single machine is illustrated, the term “machine” may also include any collection of machines that individually or jointly execute a set (or multiple sets) of instructions to perform any one or more of the methods discussed herein.

The example computing device 2100 includes a processing device 2102, a main memory 2104 (e.g., read-only memory (ROM), flash memory, dynamic random access memory (DRAM) such as synchronous DRAM (SDRAM)), a static memory 2106 (e.g., flash memory, static random access memory (SRAM)) and a data storage device 2116, which communicate with each other via a bus 2108.

Processing device 2102 represents one or more general-purpose processing devices such as a microprocessor, central processing unit, or the like. More particularly, the processing device 2102 may include a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 2102 may also include one or more special-purpose processing devices such as an application specific integrated circuit (ASIC), a field programmable gate array (FPGA), a digital signal processor (DSP), network processor, or the like. The processing device 2102 is configured to execute instructions 2126 for performing the operations and steps discussed herein.

The computing device 2100 may further include a network interface device 2122 which may communicate with a network 2118. The computing device 2100 also may include a display device 2110 (e.g., a liquid crystal display (LCD) or a cathode ray tube (CRT)), an alphanumeric input device 2112 (e.g., a keyboard), a cursor control device 2114 (e.g., a mouse) and a signal generation device 2120 (e.g., a speaker). In at least one example, the display device 2110, the alphanumeric input device 2112, and the cursor control device 2114 may be combined into a single component or device (e.g., an LCD touch screen).

The data storage device 2116 may include a computer-readable storage medium 2124 on which is stored one or more sets of instructions 2126 embodying any one or more of the methods or functions described herein. The instructions 2126 may also reside, completely or at least partially, within the main memory 2104 and/or within the processing device 2102 during execution thereof by the computing device 2100, the main memory 2104 and the processing device 2102 also constituting computer-readable media. The instructions may further be transmitted or received over a network 2118 via the network interface device 2122.

While the computer-readable storage medium 2124 is shown in an example to be a single medium, the term “computer-readable storage medium” may include a single medium or multiple media (e.g., a centralized or distributed database and/or associated caches and servers) that store the one or more sets of instructions. The term “computer-readable storage medium” may also include any medium that is capable of storing, encoding or carrying a set of instructions for execution by the machine and that cause the machine to perform any one or more of the methods of the present disclosure. The term “computer-readable storage medium” may accordingly be taken to include, but not be limited to, solid-state memories, optical media and magnetic media.

Some portions of the detailed description refer to different modules configured to perform operations. One or more of the modules may include code and routines configured to enable a computing system to perform one or more of the operations described therewith. Additionally or alternatively, one or more of the modules may be implemented using hardware including any number of processors, microprocessors (e.g., to perform or control performance of one or more operations), DSPs, FPGAs, ASICs or any suitable combination of two or more thereof. Alternatively or additionally, one or more of the modules may be implemented using a combination of hardware and software. In the present disclosure, operations described as being performed by a particular module may include operations that the particular module may direct a corresponding system (e.g., a corresponding computing system) to perform. Further, the delineating between the different modules is to facilitate explanation of concepts described in the present disclosure and is not limiting. Further, one or more of the modules may be configured to perform more, fewer, and/or different operations than those described such that the modules may be combined or delineated differently than as described.

Some portions of the detailed description are presented in terms of algorithms and symbolic representations of operations within a computer. These algorithmic descriptions and symbolic representations are the means used by those skilled in the data processing arts to convey the essence of their innovations to others skilled in the art. An algorithm is a series of configured operations leading to a desired end state or result. In example implementations, the operations carried out require physical manipulations of tangible quantities for achieving a tangible result.

Unless specifically stated otherwise, as apparent from the discussion, it is appreciated that throughout the description, discussions utilizing terms such as detecting, determining, analyzing, identifying, scanning or the like, can include the actions and processes of a computer system or other information processing device that manipulates and transforms data represented as physical (electronic) quantities within the computer system's registers and memories into other data similarly represented as physical quantities within the computer system's memories or registers or other information storage, transmission or display devices.

Example implementations may also relate to an apparatus for performing the operations herein. This apparatus may be specially constructed for the required purposes, or it may include one or more general-purpose computers selectively activated or reconfigured by one or more computer programs. Such computer programs may be stored in a computer readable medium, such as a computer-readable storage medium or a computer-readable signal medium. Computer-executable instructions may include, for example, instructions and data which cause a general-purpose computer, special-purpose computer, or special-purpose processing device (e.g., one or more processors) to perform or control performance of a certain function or group of functions.

Although the subject matter has been described in language specific to structural features and/or methodological acts, it is to be understood that the subject matter configured in the appended claims is not necessarily limited to the specific features or acts described above. Rather, the specific features and acts described above are disclosed as example forms of implementing the claims.

Unless specific arrangements described herein are mutually exclusive with one another, the various implementations described herein can be combined in whole or in part to enhance system functionality and/or to produce complementary functions. Likewise, aspects of the implementations may be implemented in standalone arrangements. Thus, the above description has been given by way of example only and modification in detail may be made within the scope of the present disclosure.

With respect to the use of substantially any plural or singular terms herein, those having skill in the art can translate from the plural to the singular or from the singular to the plural as is appropriate to the context or application. The various singular/plural permutations may be expressly set forth herein for sake of clarity. A reference to an element in the singular is not intended to mean “one and only one” unless specifically stated, but rather “one or more.” Moreover, nothing disclosed herein is intended to be dedicated to the public regardless of whether such disclosure is explicitly recited in the above description.

Terms used in the present disclosure and especially in the appended claims (e.g., bodies of the appended claims) are generally intended as “open terms” (e.g., the term “including” should be interpreted as “including, but not limited to.”).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is expressly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc.

Further, any disjunctive word or phrase preceding two or more alternative terms, whether in the description, claims, or drawings, should be understood to contemplate the possibilities of including one of the terms, either of the terms, or both of the terms. For example, the phrase “A or B” should be understood to include the possibilities of “A” or “B” or “A and B.”

Additionally, the use of the terms “first,” “second,” “third,” etc., are not necessarily used herein to connote a specific order or number of elements. Generally, the terms “first,” “second,” “third,” etc., are used to distinguish between different elements as generic identifiers. Absence a showing that the terms “first,” “second,” “third,” etc., connote a specific order, these terms should not be understood to connote a specific order. Furthermore, absence a showing that the terms first,” “second,” “third,” etc., connote a specific number of elements, these terms should not be understood to connote a specific number of elements.

All examples and conditional language recited in the present disclosure are intended for pedagogical objects to aid the reader in understanding the present disclosure and the concepts contributed by the inventor to furthering the art, and are to be construed as being without limitation to such specifically recited examples and conditions. Although embodiments of the present disclosure have been described in detail, various changes, substitutions, and alterations could be made hereto without departing from the spirit and scope of the present disclosure.

What is claimed is:

1. A token adjustment system, comprising:
a carbon conversion device operable to generate a syngas from a greenhouse gas;
a processing device operable to:
receive one or more measurements, wherein the one or more measurements comprise one or more of an input flow measurement received from an input flow sensor, an output flow measurement received from an output flow sensor, an energy measurement from an energy sensor, or a utility measurement received from a utility sensor;
compute one or more of a carbon balance, a carbon intensity, or a carbon adjustment based on the one or more measurements; and
cause a token to be generated or consumed based on the one or more of the carbon balance, the carbon intensity, or the carbon adjustment.

2. The token adjustment system of claim 1, wherein the token is generated to have a token value based on the one or more of the carbon balance, the carbon intensity, or the carbon adjustment.

3. The token adjustment system of claim 1, wherein the one or more of the carbon balance, the carbon intensity, or the carbon adjustment is computed based on one or more of a total carbon input, a total carbon output, an energy, or a utility used for carbon reduction.

4. The token adjustment system of claim 1, wherein the one or more of the carbon balance, the carbon intensity, or the carbon adjustment is computed based on one or more of a carbon input flow rate, a carbon output flowrate, an energy, or a utility used for carbon reduction.

5. The token adjustment system of claim 1, wherein the one or more of the carbon intensity or the carbon adjustment is computed based on the carbon balance.

6. The token adjustment system of claim 1, wherein the greenhouse gas is one or more of CO₂ or CH₄.

7. The token adjustment system of claim 1, wherein the syngas is generated at a ratio of from about 0.5:1 of H₂:CO to about 3:1 of H₂:CO.

8. The token adjustment system of claim 1, further comprising:
a syngas transformation device operable to generate one or more decarbonized products from the syngas.

9. The token adjustment system of claim 8, wherein the token has a token value based on the one or more decarbonized products.

10. The token adjustment system of claim 8, wherein the one or more decarbonized products are one or more of methanol, ethanol, acids, ammonia, hydrogen, or sustainable aviation fuel.

11. The token adjustment system of claim 1, wherein the token is validated using location data.

12. A method for adjusting tokens, comprising:
detecting one or more measurements comprising one or more of an input flow measurement received from an input flow sensor, an output flow measurement received from an output flow sensor, an energy measurement from an energy sensor, or a utility measurement received from a utility sensor;
computing one or more of a carbon balance, a carbon intensity, or a carbon adjustment based on the one or more measurements; and
causing a token to be adjusted based on the one or more of the carbon balance, the carbon intensity, or the carbon adjustment.

13. The method of claim 12, further comprising:
generating the token when the one or more of the carbon balance, the carbon intensity, or the carbon adjustment is a carbon reduction.

14. The method of claim 12, further comprising:
consuming the token when the one or more of the carbon balance, the carbon intensity, or the carbon adjustment is a carbon increase.

15. The method of claim 12, further comprising:
computing one or more of the carbon intensity or the carbon adjustment using one or more of a carbon balance computation or a carbon intensity computation.

16. The method of claim 12, further comprising:
computing the carbon adjustment relative to a baseline process.

17. The method of claim 12, further comprising:
validating the token using location data.

18. A device operable to adjust tokens, comprising:
a sensor operable to measure one or more metrics comprising one or more of an environmental input metric, an environmental output metric, or an environmental energy metric;
a processing device operable to:
compute an environmental effect based on the one or more metrics; and
cause a token to be adjusted based on the environmental effect.

19. The device of claim 18, wherein the processing device is operable to cause the token to be generated when the environmental output metric is greater than a combined total of the environmental input metric and the environmental energy metric.

20. The device of claim 18, wherein the processing device is operable to cause the token to be consumed when the environmental output metric is less than a combined total of the environmental input metric and the environmental energy metric.

21. The device of claim 18, wherein the token is adjusted to a token value based on an environmental effect metric of the environmental effect.

22. The device of claim 18, wherein the processing device is operable to validate the token using location data.
